(19) United States
(12) Patent Application Publication
(10) Pub. No.: US 2026/0112036 A1
(43) Pub. Date: Apr. 23, 2026

(54) DEVICES, SYSTEMS, AND METHODS FOR NEUROMODULATION

(71) Applicants: Washington University, St. Louis, MO (US); Regents of the University of Minnesota, Minneapolis, MN (US)

(72) Inventors: Nico DOSENBACH, St. Louis, MO (US); Evan GORDON, St. Louis, MO (US); Damien FAIR, Minneapolis, MN (US); Timothy LAUMANN, St. Louis, MO (US)

(21) Appl. No.: 19/118,075

(22) PCT Filed: Oct. 26, 2023
(86) PCT No.: PCT/US2023/077873

§ 371 Date: Apr. 3, 2025

Related U.S. Application Data
(60) Provisional application No. 63/419,696, filed on Oct. 26, 2022.

(51) Int. Cl. G06T 7/00; A61B 5/00; A61B 5/055; A61M 5/172; A61N 1/36; G01R 33/48; G06T 7/12; G06T 7/73; G16H 20/70

(52) U.S. Cl. CPC G06T 7/0016; A61B 5/0042; A61B 5/055; A61B 5/4848; A61B 5/7275; G01R 33/4806; G06T 7/12; G06T 7/73; G16H 20/70; A61B 2576/026; A61M 5/1723; A61N 1/36135; G06T 2207/10088; G06T 2207/30016

(57) ABSTRACT

System and methods for mapping a network in a brain of a subject is provided. The method includes receiving task-based functional magnetic resonance imaging (fMRI) data of a brain of a subject, receiving resting-state fMRI data of the brain, locating effector-specific regions of the brain and inter-effector regions of the brain based on the task-based fMRI data and the resting-state fMRI data, and identifying a network in the brain based on functional connectivity with the inter-effector regions based on the resting-state fMRI data to then generate one or more maps of the network.

### CROSS-REFERENCE TO RELATED APPLICATIONS

This application claims priority to and the benefit of U.S. Provisional Patent Application Ser. No. 63/419,696, entitled “DEVICES, SYSTEMS, AND METHODS RELATED TO A MIND-BODY INTERFACE (MBI) CIRCUIT FOR NEUROMODULATION,” filed Oct. 26, 2022, and the contents of which are incorporated herein in their entirety.

### STATEMENT REGARDING FEDERALLY SPONSORED RESEARCH OR DEVELOPMENT

This invention was made with government support under MH096773, MH124567, MH121276, and MH122066 awarded by the National Institutes of Health. The government has certain rights in the invention.

### FIELD OF THE INVENTION

The present disclosure generally relates to devices, systems, and methods for neuromodulation in neurological and psychiatric disorders and brain injury from any source.

### BACKGROUND OF THE INVENTION

Patient-specific precision and accuracy are critically important for successful neuromodulation, both for established (e.g., deep brain stimulation (DBS) of ventralis intermedius (VIM) nucleus of the thalamus) and investigational (e.g., DBS of centromedian (CM) nucleus) clinical targets. The current standard of care for FDA-cleared neuromodulation—be it DBS, focused US for movement disorders (VIM, GPi, STN), or TMS for depression (dlPFC)—relies on very simple one-size-fits-all clinical consensus coordinates for targeting. Several recent studies have suggested that advanced imaging driven (e.g., DTI, functional connectivity) patient-specific targeting might improve outcomes while simultaneously improving procedural efficiency. In addition, a better neurophysiological understanding of the targeted structures and their inter-connectivity in individual patients is vital for selecting the most appropriate of multiple candidate targets for intervention, for assessing and/or predicting treatment response, for identifying the relatively best target within a circuit (for a given patient), and for identifying and verifying novel targets anywhere in the brain. The currently poor understanding of the neurophysiology underlying successful neuromodulation for brain disorders hinders the ability to match patients with the most appropriate therapy and slows the progression of patients toward potentially life-altering neuromodulation.

### SUMMARY OF THE INVENTION

Among the various aspects of the present disclosure is the provision of devices, systems, and methods related to the identification and characterization of targets for clinical treatment of a brain of a subject.

In one aspect, a system for mapping a network in a brain of a subject is provided. The system includes at least one processor in communication with at least one memory device. The at least one processor is programmed to receive task-based functional magnetic resonance imaging (fMRI) data of a brain of a subject, receive resting-state fMRI data of the brain, locate effector-specific regions of the brain and inter-effector regions of the brain based on the task-based fMRI data and the resting-state fMRI data, identify a network in the brain based on functional connectivity with the inter-effector regions based on the resting-state fMRI data, and generate one or more maps of the network.

In another aspect, a method of identifying a treatment is provided. The method includes receiving task-based fMRI data of a brain of a subject, receiving resting-state fMRI data of a brain of a subject, locating effector-specific regions of the brain and inter-effector regions of the brain based on the task-based fMRI data and the resting-state fMRI data, identifying a network based on functional connectivity with the inter-effector regions based on the resting-state fMRI data, generating one or more maps of the network, and selecting a treatment based on the one or more maps of the network.

The present teachings include devices and systems for a Mind-Body Interface (MBI). In some aspects, the MBI identifies inter-connected brain regions. In some aspects, the MBI device includes an MBI interface system, an MRI system, and at least one computing system. In some aspects, the MBI system identifies inter-connected Brain regions. In some aspects, the MBI system includes an MRI system and at least one computing device.

The present teachings include methods for improving treatment for neuropsychiatric symptoms, disorders, or brain injury in a patient-specific manner using a Mind-Body Interface identification system to identify a set of interconnected brain regions. In some aspects, the Mind-Body Interface includes a Mind-Body Interface system, an MR system, and at least one computing device. In some aspects, the treatment can be invasive or non-invasive neuromodulation or ablative techniques. In another aspect, the functional status and response to the treatment can be assessed by the MBI system. In some aspects, the interconnected brain regions can be identified using precision functional mapping (PFM) functional connectivity methods applied to BOLD data from all brain states (e.g., resting, task, movie watching, asleep, sedated). In yet another aspect, the BOLD data can be further annotated using structural metrics (e.g., cortical thickness) and DTI. In another aspect, differences in relative functional connectivity at baseline can be used to triage patients into the most appropriate therapies. In an aspect, changes in functional connectivity in response to various treatments, including neuromodulation, can be used to assess and further refine treatment parameters. In another aspect, the MBI enables actual implementation of behavior, as expressed through movement and physiological changes, and therefore allows superior, more behavior-targeted interventions than targeting more brain regions associated with more abstract functions (e.g., dorsolateral PFC). In some embodiments, the interconnected brain regions can be well circumscribed in the cortex, basal ganglia, thalamus, brainstem, and cerebellum. In some embodiments, the method enables a systematic search for the most effective, most reliable and safest nodes within it to target for any given condition, symptom, or injury type. In some embodiments, the centromedian nucleus of the thalamus can be identified. In some embodiments, the patient has generalized epilepsy, chronic pain, Tourette's, or Parkinson's disease.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A is a block diagram schematically illustrating a computing device in accordance with one aspect of the disclosure.

FIG. 1B is a flowchart illustrating a method of identifying a treatment;

FIG. 1C is a flowchart illustrating a method of generating maps, assessing connectivity, and/or selecting or evaluating treatment in accordance with some aspects of the present disclosure;

FIG. 2 is a block diagram schematically illustrating a system in accordance with one aspect of the disclosure;

FIG. 3 is a block diagram schematically illustrating a remote or user computing device in accordance with one aspect of the disclosure;

FIG. 4 is a block diagram schematically illustrating a server system in accordance with one aspect of the disclosure;

FIG. 5A depicts resting state functional connectivity (RSFC) seeds along a continuous line of cortical locations in the left precentral gyrus in a single exemplar participant;

FIG. 5B depicts RSFC seeded from a continuous line of cortical locations in the left precentral gyrus as shown in FIG. 5A in a single exemplar participant;

FIG. 6 depicts RSFC seeded from a continuous line of cortical locations in the left precentral gyrus for an exemplar group of highly-sampled participants;

FIG. 7 depicts RSFC seeded from a continuous line of cortical locations in the left precentral gyrus for within-participant replications;

FIG. 8 depicts RSFC seeded from a continuous line of cortical locations in the left precentral gyrus for group-averaged data;

FIG. 9 depicts discrete functional networks demarcated using a whole-brain, data-driven, hierarchical approach applied to the resting-state fMRI data, which defined the spatial extent of the networks;

FIG. 10 depicts discrete functional networks demarcated using a whole-brain, data-driven, hierarchical approach applied to the resting-state fMRI data for other participants;

FIG. 11 depicts an example of how the inter-effector connectivity pattern became more distinct from surrounding effector-specific motor regions as connectivity thresholding increased from the 80th to the 97th percentile;

FIG. 12A depicts functional connectivity maps seeded from a continuous line of points down precentral gyrus in fMRI data from data averaged across 262 human neonates, all scanned shortly after birth;

FIG. 12B depicts functional connectivity maps seeded from a continuous line of points down precentral gyrus in fMRI data from a neonate scanned 13 days after birth;

FIG. 12C depicts functional connectivity maps seeded from a continuous line of points down precentral gyrus in fMRI data from an 11-month old infant;

FIG. 12D depicts functional connectivity maps seeded from a continuous line of points down precentral gyrus in fMRI data from a 9-year old child;

FIG. 12E depicts functional connectivity maps seeded from a continuous line of points down precentral gyrus in fMRI data from adult participant P01;

FIG. 12F depicts functional connectivity maps seeded from a continuous line of points down precentral gyrus in fMRI data from an adolescent who had experienced extensive cortical reorganization after severe bilateral perinatal strokes;

FIG. 13 depicts brain regions with the strongest functional connectivity to the left middle inter-effector region in cortex, striatum, thalamus, and cerebellum in the exemplar participant;

FIG. 14 depicts brain regions with the strongest functional connectivity to the left middle inter-effector region in cortex, striatum, thalamus, and cerebellum in the other participants;

FIG. 15 depicts brain regions more strongly functionally connected to inter-effectors than to any foot/hand/mouth regions from adult participant P01;

FIG. 16A depicts brain regions with the strongest functional connectivity to the middle inter-effector region in medial cortex;

FIG. 16B depicts brain regions with the strongest functional connectivity to the middle inter-effector region in striatum;

FIG. 16C depicts brain regions with the strongest functional connectivity to the middle inter-effector region in thalamus;

FIG. 16D depicts brain regions with the strongest functional connectivity to the middle inter-effector region in cerebellum;

FIG. 17 depicts inter-network relationships visualized in network space using a spring-embedding plot;

FIG. 18A depicts inter-effector vs effector specific functional connectivity for all participants;

FIG. 18B depicts inter-network relationships visualized in network space using a spring-embedding plot for all participants;

FIG. 19 depicts inter-effector and effector-specific regions tested for systematic differences in the temporal ordering of their infra-slow fMRI signals;

FIG. 20 depicts inter-effector regions in relation to cortical thickness;

FIG. 21 depicts cerebellum connectivity and task activation;

FIG. 22A depicts functional connectivity strength between M1 region and individual-specific cingulo-opercular network;

FIG. 22B depicts functional connectivity strength between M1 region and middle insula;

FIG. 22C depicts functional connectivity strength with Lobule VIIIa vermis of the cerebellum;

FIG. 22D depicts functional connectivity strength between M1 region and dorsal posterior putamen;

FIG. 22E depicts functional connectivity strength between M1 region and nuclei of the thalamus: ventral Intermediate nucleus;

FIG. 22F depicts functional connectivity strength between M1 region and the parafascicular nucleus;

FIG. 22G depicts functional connectivity strength between M1 region and the ventral lateral anterior nucleus;

FIG. 22H depicts functional connectivity strength between M1 region and adjacent postcentral gyrus;

FIG. 22I depicts cortical thickness in M1 region;

FIG. 22J depicts fractional anisotropy within 2 mm below cortex under M1 region;

FIG. 22K depicts relative myelin density in M1 region;

FIG. 23 depicts task fMRI activations during a movement task battery, including movement of the toes, ankles, knees, gluteus, abdominals, shoulders, elbows, hands, eyebrows, eyelids, tongue, and swallowing;

FIG. 24 depicts activation strength for each movement was computed along the dorsal-ventral axis within M1;

FIG. 25 depicts fMRI activations for somatomotor-hand and Brodmann areas 1-4 for all participants;

FIG. 26 depicts fMRI activations for inter-effector network and Brodmann areas 1-4 for all participants;

FIG. 27 depicts inter-effector regions co-activated during abdominal contraction;

FIG. 28 depicts inter-effector regions activity during movements;

FIG. 29 depicts event-related task fMRI data during an action planning task with separate planning and execution phases for movements of the hands and feet;

FIG. 30 depicts activation profiles with one- and two-peak fits;

FIG. 31 depicts activation with two-peak curves;

FIG. 32 depicts tasks for each participant;

FIG. 33 depicts coactivation with CON;

FIG. 34A depicts functional connectivity maps were seeded from points in precentral gyrus in a macaque;

FIG. 34B depicts a schematic illustrating proposed analogous regions in macaque and in human;

FIG. 35 depicts Penfield's classical homunculus depicting a continuous map of the body in primary motor cortex;

FIG. 36 depicts the integrate-isolate model of primary motor cortex organization, effector-specific zones represented by concentric rings with proximal body parts surrounding the relatively more isolatable distal ones;

FIG. 37 depicts motor stimulations mapped onto cortex; and

FIG. 38 depicts an overview of the action control and coordination task.

### DETAILED DESCRIPTION OF THE INVENTION

The present disclosure is based, at least in part, on the discovery that the Mind-Body Interface described herein can greatly improve the ability to treat a variety of neuropsychiatric symptoms, disorders, and brain injuries.

Our discovery and characterization of a previously unrecognized, distributed brain system for integrating abstract behavioral plans with movements and autonomic functions of the body. As described herein, this may be referred to as the Mind-Body Interface (MBI) or Somato-Cognitive Action Network (SCAN). The system and methods provided herein are configured to identify neuromodulation targets, facilitate advanced imaging-based methods for identifying novel and established targets, support triaging patients to the most appropriate therapy, and provide a basis for assessing treatment response via patient-specific imaging.

Among the nodes of the MBI (or SCAN) are the supplementary motor area (SMA), the middle insula, the newly described inter-effector regions in the primary motor cortex (M1), the dorsal putamen, the VMI and CM nuclei of the thalamus, the red nucleus, the STN, the substantia nigra, and the dorsal motor nucleus of the vagus nerve. Advanced precision functional mapping (PFM) functional connectivity (FC) analyses can be used to identify and characterize the MBI as a whole, as well as each of these individual nodes and their functional subdivisions.

The MBI represents a third brain system important for initiating and controlling movements, in addition to the eye-movement circuitry and the classical circuitry for controlling highly dexterous effectors (the feet/toes, hands/fingers, and mouth/tongue). In addition to holistic, whole-body motor control, the MBI also carries downstream information about action plans and behavioral goals, and prepares the body physiologically for upcoming activity via direct control over the autonomic nervous system (e.g., adrenal medulla). Since arousal is a pre-requisite to successful action, the MBI also carries critically important arousal/vigilance information. Furthermore, the MBI receives and processes feedback important for controlling real-world actions, such as posture, pain, and visceral sensations. Existing neuromodulation already targets different nodes of the MBI, such as the VIM for Essential Tremor and Parkinson's Disease (PD) and the CM for generalized epilepsy, Tourette's, pain, Parkinson's, and disorders of consciousness. Vagus nerve stimulation procedures in use for generalized epilepsy and under investigation for depression may also modulate activity within the MBI via the dorsal motor nucleus of the vagus nerve and the CM.

Given the functions of the MBI, modulating its activity could also be used to treat apathy, abulia, and akinetic mutism due to various disorders or injuries, in addition to tremors (global motor control), generalized seizures (arousal), chronic pain, and disorders of consciousness (arousal). Given that the MBI also controls autonomic effects, secondary to abstract ideas and plans, it might also be targeted for panic and anxiety disorders.

One aspect of the present disclosure provides for devices, systems, and methods related to the Mind-Body Interface.

The technology to identify a set of inter-connected brain regions named the Mind-Body Interface (MBI), in a patient-specific manner, using advanced brain MRI methods is described. A method is described to assess their functional status and response to any type of intervention, including the use of invasive and non-invasive neuromodulation, as well as ablative techniques is also described, which can greatly improve the ability to treat a variety of neuropsychiatric symptoms, disorders, and brain injuries. The MBI nodes can be identified using novel precision functional mapping (PFM) functional connectivity methods applied to BOLD data from all brain states (e.g., resting, task, movie watching, asleep, sedated). They can be further annotated using structural metrics (e.g., cortical thickness) and DTI. Differences in relative functional connectivity at baseline can be used to triage patients into the most appropriate therapies, and changes in functional connectivity in response to various treatments, including neuromodulation, can be used to assess and further refine treatment parameters. The MBI enables actual implementation of behavior, as expressed through movement and physiological changes, and therefore allows superior, more behavior-targeted interventions than targeting more brain regions associated with more abstract functions (e.g., dorsolateral PFC). In addition, the MBI system's specific nodes are well circumscribed in the cortex, basal ganglia, thalamus, brainstem, and cerebellum, making them more precise targets. The understanding of the functions of the MBI enables a systematic search for the most effective, most reliable and safest nodes within it to target for any given condition, symptom, or injury type. For example, the methods for identifying the MBI enables precise and accurate identification of the CM (centromedian nucleus) of the thalamus in individuals. This is of great clinical importance because the CM is being studied as a potential target in generalized epilepsy, chronic pain, Tourette's, and Parkinson's, yet there are currently no accepted consensus clinical coordinates for identifying the CM, and there are no established, standardly available methods for identifying it from structural MRI data.

### Computing Systems and Devices

In various aspects, the disclosed MBI methods may be implemented using a computing system or computing device.

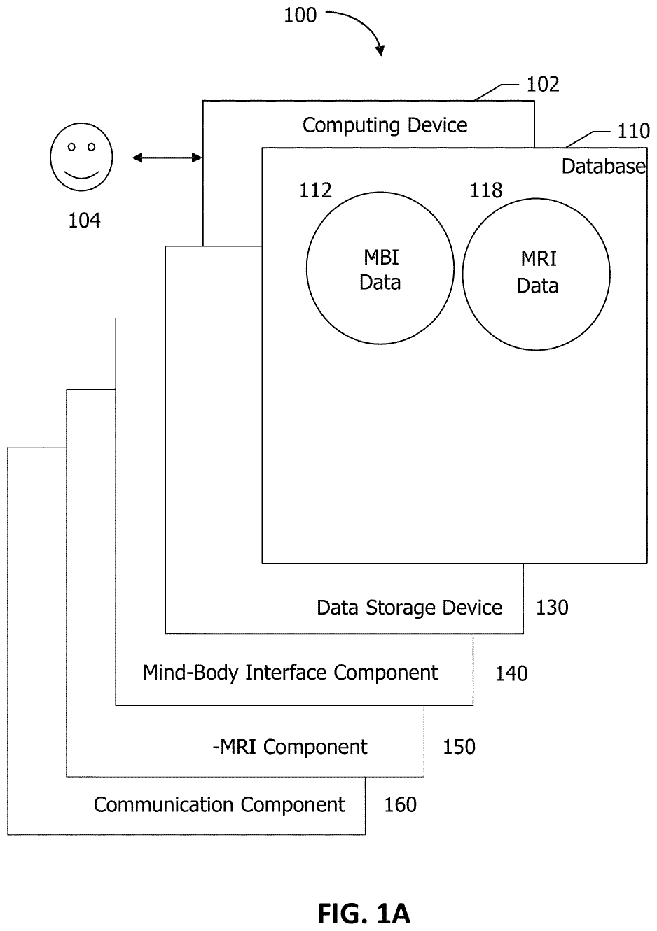

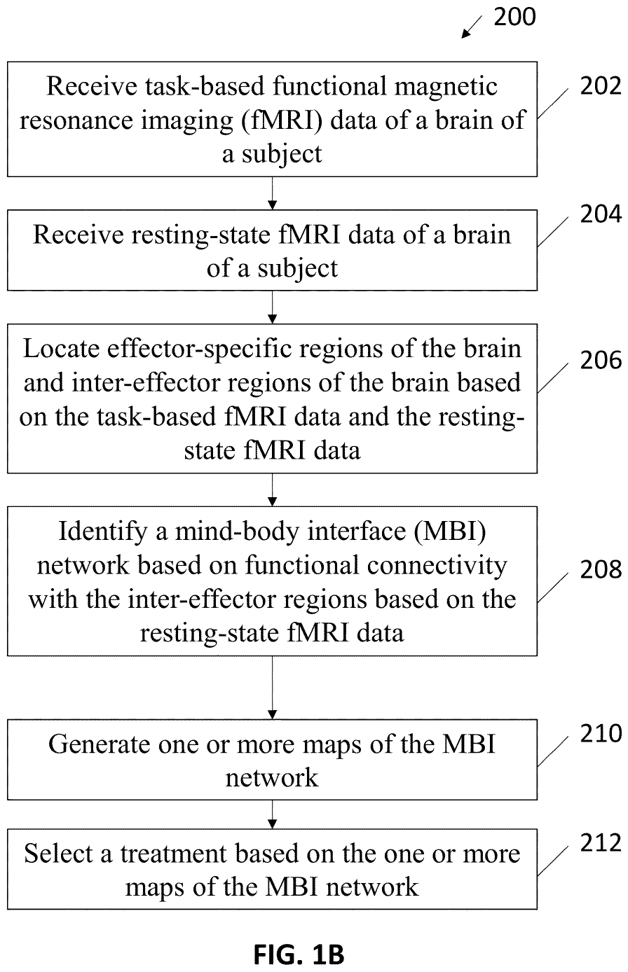

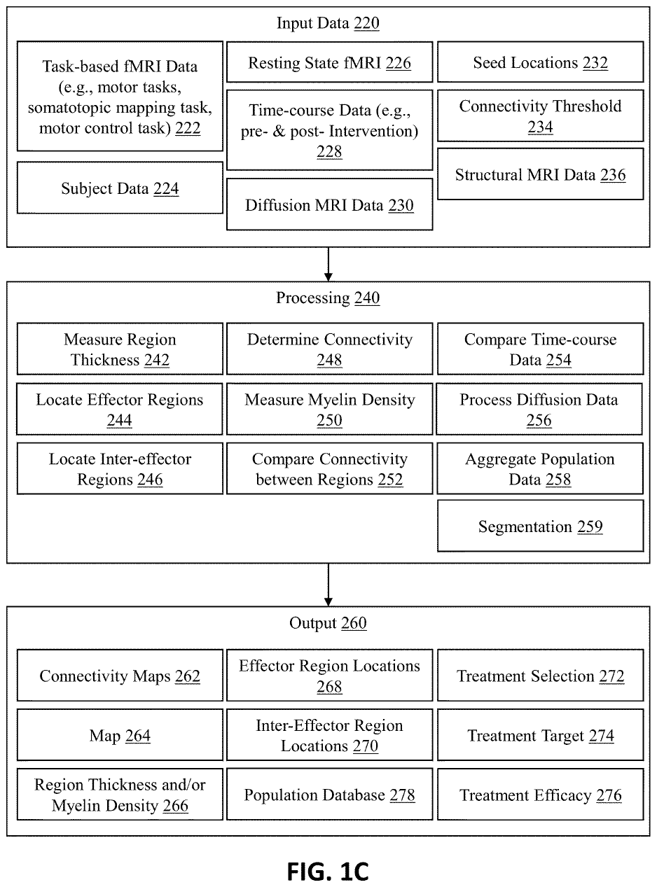

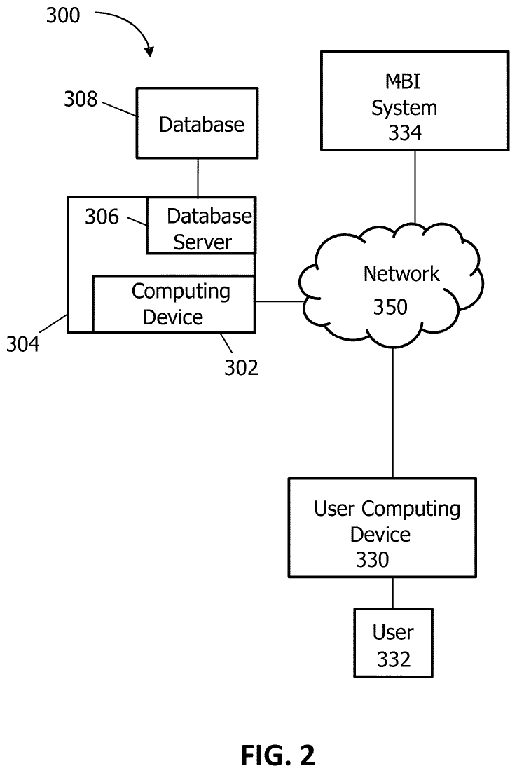

FIG. 1A depicts a component configuration 100 of computing device 102, which includes database 110 along with other related computing components. In some aspects, computing device 102 is similar to computing device 302 (shown in FIG. 2). A user 104 may access components of computing device 102. In some aspects, database 110 is similar to database 308 (shown in FIG. 2).

In one aspect, database 110 includes MBI data 112 and MRI data 118. MBI data 112 may include data used to operate an MBI system using MRI data. Non-limiting examples of MBI data 112 include various MRI data, any parameters used to control the operation of an MBI device, and any parameters defining equations or other algorithms used to implement the MBI system as disclosed herein. MRI data 118 may include data used to perform the methods implementing the MBI system or devices as disclosed herein. Non-limiting examples of MRI data 118 include measurements of background noise or MRI signals, any parameters defining equations and other algorithms used to implement the transformation of background noise and MRI signals into differential MRI signals as disclosed herein and/or any parameters defining equations and other algorithms used to implement MBI method described herein.

Computing device 102 also includes a number of components that perform specific tasks. In the exemplary aspect, computing device 102 includes a data storage device 130, an MBI component 140, an MRI component 150, and a communication component 160. The MBI component 140 is configured to implement the MBI method as described herein. The MRI component 150 is configured to implement the MBI methods from MRI data as disclosed herein. The data storage device 130 is configured to store data received or generated by computing device 102, such as any of the data stored in database 110 or any outputs of processes implemented by any component of computing device 102.

The communication component 160 is configured to enable communications between computing device 102 and other devices (e.g. user computing device 330 shown in FIG. 2) over a network, such as a network 350 (shown in FIG. 2), or a plurality of network connections using predefined network protocols such as TCP/IP (Transmission Control Protocol/Internet Protocol).

Functional magnetic resonance imaging (fMRI) has been used to study and diagnose brain cognitive functions and neural connectivity. During resting-state fMRI or resting state MRI, the subject is at rest. The subject lies in the MR scanner for a period of time awake or sedated while MRI data are acquired. During task-based fMRI, the subject performs tasks while MRI data are acquired.

FIG. 1B depicts a method 200 of identifying a treatment. The method 200 includes receiving 202 task-based fMRI data of a brain of a subject, receiving 204 resting-state fMRI data of a brain of a subject, locating 206 effector-specific regions of the brain and inter-effector regions of the brain based on the task-based fMRI data and the resting-state fMRI data, identifying 208 a mind-body interface (MBI) network based on functional connectivity with the inter-effector regions based on the resting-state fMRI data, generating 210 one or more maps of the MBI network, and selecting 212 a treatment based on the one or more maps of the MBI network.

Referring to FIG. 1C, a method is illustrated that may be used to generate network maps, assess inter-effector region connectivity, and/or provide treatment information or guidance. The method may be carried out by a computer system or by a computer system with input from a user, such as a clinician. For example, the computer system may receive input data 220. The input data may include fMRI data, which may be accessed from a magnetic resonance imaging (MRI) system or from a computer memory. The fMRI data may include task-based fMRI data 222, which may be acquired while a subject conducts a motor task, such as a motor control task or somatotopic mapping task. The fMRI data may also include resting state fMRI 226, such as eyes closed resting state, eyes open resting state, or eyes fixated resting state data.

The input data 220 may also include diffusion MRI data 230, such as diffusion weighted imaging data or diffusion tensor imaging data, and structural MRI data 236. The input data 220 may include data from a single time point or a series of time-course data 228. For example, the fMRI data may be acquired before and after an intervention (e.g., treatment, clinical intervention, immobilization intervention, drug administration, etc.) or before and after a clinical event (e.g., stroke). The imaging data may also be augmented with subject data 224, such as clinical or demographic data. For example, the subject data 224 may include subject age, clinical history, diagnosis, treatment history, subject's dominant hand, etc.

The input data 220 may also include processing parameters. For example, a user may define or choose seed locations 232 for processing 240 or may choose or adjust a connectivity threshold 234 based on the data, subject, desired application, etc.

The method also includes processing the input data 220 in block 240, which may be carried out by a computer system with or without input from a user. Processing 240 may be performed on individual subject data, across data acquired at multiple time points, or using data grouped based on population characteristics.

Processing 240 may include determining functional connectivity 248 using the fMRI data (e.g., 226, 228). As a non-limiting example, connectivity may be estimated using a vertex/voxel-wise functional connectivity matrix calculated from fMRI data using a Fisher-transformed pairwise correlation of the timeseries of the vertices/voxels in the brain. Other methods for determining functional connectivity may also be used. Determining functional connectivity 248 may also include identifying seed points. As a non-limiting example, a line of seeds may be defined down the precentral gyrus. The connectivity of each seed point may be analyzed using Fisher-transformed correlation between the time course of each seed point and the time course of the other voxels within the brain. In this way, processing 240 may also include manual, semi-automatic, or automatic (e.g., using Freesurfer) segmentation 259 of the MRI data. Segmentation 259 may include surface segmentation, surface delineation, and atlas registration. As a non-limiting example, segmentation may be performed using a T2-weighted image, and may be co-registered to the other structural, functional, and diffusion MRI images.

Processing 240 may also include identifying brain regions, including locating effector regions 244 and locating inter-effector regions 246. Such regions may be identified using the functional connectivity data. As a non-limiting example, a data-driven network detection algorithm may be used to identify network subdivisions. Other methods of region of interest analysis may also be used, which may include use of atlas data or machine learning algorithms, for example. In some implementations, inter-effector subnetworks may be manually or automatically grouped together into one network structure for further processing. Identified brain regions may be defined or visualized with respect to known or pre-defined brain regions (e.g., Brodmann areas).

Processing 240 may also include extracting other measurements and parameters related to connectivity. Such measures may be compared between regions, such as between specific effector regions (e.g., foot, hand, mouth) or between effector regions and inter-effector regions. As one example, cortical thickness may be measured based on segmentation data, which may be deformed and registered to functional data. In this way, the thickness of each region may be measured and compared. For example, thickness may be compared between effector regions and inter-effector regions using a paired t-test across multiple subjects. As another example, processing 240 may include measuring myelin density 250 within each identified region. Processing 240 may also include processing diffusion data 256, such as generating fractional anisotropy maps or diffusion tensor images. As a non-limiting example, fractional anisotropy may be compared between identified regions, such as between effector regions and inter-effector regions.

The resulting data may be further processed 240 to compare outputs. For example, processing 240 may include comparing results (e.g., connectivity, effector region and inter-effector region locations, region thickness, myelin density, etc.) across different time points 254 (e.g., pre- and post-intervention) for a given subject. Connectivity between regions may also be measured and compared 252. For example, the connectivity may be measured between effector regions with other brain regions (e.g., CON, adjacent postcentral gyrus, middle insula, cerebellum, putamen, thalamus, etc.), and between inter-effector regions with other brain regions. Such functional connectivity may be compared between effector regions and inter-effector regions, for example. The resulting data may also be grouped by patient population 258 and compared across various patient populations (e.g., age group, patient who have or have not experience brain injury or stroke, etc.).

Several types of outputs 260 may be provided by the method to generally characterize functional connectivity, brain regions, and treatment information. For example, the output 260 may include connectivity maps 262, which may provide a visual representation of overall connectivity or localized connectivity of effector regions and inter-effector regions. The output 260 may also include a network map 264 that provides visualization of inter-effector and effector regions. The output may also include coordinates for locations of effector regions 268 and inter-effector regions 270, which may be visualized in conjunction with connectivity data, structural MRI images, diffusion images, etc. The output 260 may also include a report or visualization or region thickness and/or myelin density 266, for example or each identified region. The output 260 may also provide one or more population databases 278. For example, an atlas of brain connectivity and effector and inter-effector region locations may be produced for a given patient population (e.g., infant, pediatric, adult, PD patient, pre- and post-treatment etc.).

The output 260 may also include treatment information to guide treatment selection, target, or evaluation. For example, the output 260 may include a treatment selection 272 based in part on other output data 260. Treatment selection 272 may include the selection of a drug, invasive neuromodulation, non-invasive neuromodulation, or other intervention. For example, treatment selection 272 may include selection of deep brain stimulation (DBS) parameters. Treatment selection may be determined by a trained clinician, guided by the output data 260 provided by the method. In other configurations, treatment selection may be performed by an automated algorithm generated or trained based on population data, subject data, and outcome data. The maps may also be used to identify a localized treatment target 274 for neuromodulation (e.g., DBS placement). The output 260 may also be used to evaluate treatment efficacy 276. For example, functional connectivity may be assessed before and after treatment to evaluate connectivity changes achieved by treatment.

FIG. 2 depicts a simplified block diagram of the system for implementing the computer-aided method described herein. As illustrated in FIG. 2, the computing device 300 may be configured to implement at least a portion of the tasks associated with the disclosed methods described herein. The computer system 300 may include a computing device 302. In one aspect, the computing device 302 is part of a server system 304, which also includes a database server 306. The computing device 302 is in communication with a database 308 through the database server 306. The computing device 302 is communicably coupled to a user computing device 330 and a Mind-Body Interface (MBI) system 334 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

In other aspects, the computing device 302 is configured to perform a plurality of tasks associated with the disclosed computer-aided methods using the MBI circuit. In some aspects, the computing device 302, user computing device 330, and/or MBI system 334 may be operatively connected via a network 350.

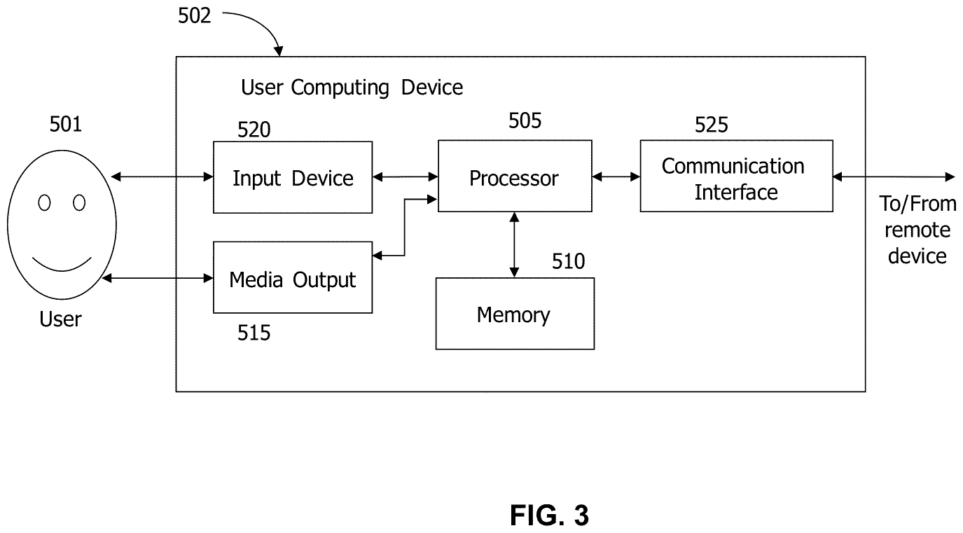

FIG. 3 depicts a configuration of a remote or user computing device 502, such as user computing device 330 (shown in FIG. 2). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

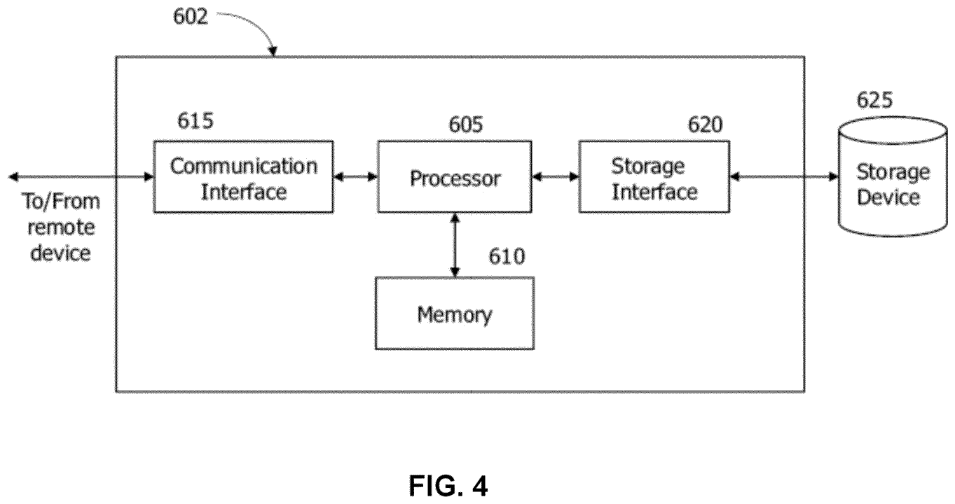

FIG. 4 illustrates an example configuration of a server system 602. Server system 602 may include, but is not limited to, database server 306 and computing device 302 (both shown in FIG. 2). In some aspects, server system 602 is similar to server system 304 (shown in FIG. 2). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 2) or another server system 602. For example, communication interface 615 may receive requests from a user computing device 330 via a network 350 (shown in FIG. 2).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for the storage of a computer program.

The computer systems and computer-aided methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

The methods and algorithms of the disclosure may be enclosed in a controller or processor. Furthermore, methods and algorithms of the present disclosure, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a region within a medical image (segmentation), categorization of a type of motion, a diagnosis based on the motion of an object, motion analysis of an object, and trained model parameters ML outputs may further include: speech recognition, image or video recognition, medical diagnoses, statistical or financial models, autonomous vehicle decision-making models, robotics behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: genetic algorithms, linear or logistic regressions, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, adversarial learning, and reinforcement learning.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

### Therapeutic Methods

Also provided is a process of treating, preventing, or reversing neuropsychiatric symptoms, disorders, or brain injuries in a subject in need of administration of a therapeutically effective amount of neurological treatment, so as to improve neuropsychiatric symptoms, disorders, and brain injuries.

Methods described herein are generally performed on a subject in need thereof. A subject in need of the therapeutic methods described herein can be a subject having, diagnosed with, suspected of having, or at risk for developing neuropsychiatric symptoms, disorders, or brain injuries. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of neurological treatment is, for example, an amount that would cause the desired therapeutic effect in a subject while minimizing undesired side effects. In various embodiments, an effective amount of a neurological treatment described herein can substantially inhibit neuropsychiatric symptoms, disorders, or brain injuries, slow the progress of neuropsychiatric symptoms, disorders, or brain injuries, or limit the development of neuropsychiatric symptoms, disorders, or brain injuries

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of neurological treatment can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to improve neuropsychiatric symptoms, disorders, and brain injuries.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a neurological treatment can occur as a single event or over a time course of treatment. For example, a neurological treatment can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for a neurological disease.

A neurological treatment can be administered simultaneously or sequentially with another agent, such as an antibiotic, an anti-inflammatory, or another agent. For example, a neurological treatment can be administered simultaneously with another agent, such as an antibiotic or an anti-inflammatory. Simultaneous administration can occur through the administration of separate compositions, each containing one or more of a neurological treatment, an antibiotic, an anti-inflammatory, or another agent. Simultaneous administration can occur through the administration of one composition containing two or more of a neurological treatment, an antibiotic, an anti-inflammatory, or another agent. A neurological treatment can be administered sequentially with an antibiotic, an anti-inflammatory, or another agent. For example, a neurological treatment can be administered before or after the administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Summary

Primary motor cortex (M1) has been thought to form a continuous somatotopic homunculus extending down precentral gyrus from foot to face representations. The motor homunculus has remained a textbook pillar of functional neuroanatomy, despite evidence for concentric functional zones and maps of complex actions. Using our highest precision functional magnetic resonance imaging (fMRI) data and methods, it was discovered that the classic homunculus is interrupted by regions with sharpy distinct connectivity, structure, and function, alternating with effector-specific (foot, hand, mouth) areas. These inter-effector regions exhibit decreased cortical thickness and strong functional connectivity to each other, and to prefrontal, insular, and subcortical regions of the Cingulo-opercular network (CON), critical for executive action and physiological control, arousal, and processing of errors and pain. This inter-digitation of action control-linked and motor effector regions was independently verified in the three largest fMRI datasets. Macaque and pediatric (newborn, infant, child) precision fMRI revealed potential cross-species analogues and developmental precursors of the inter-effector system. An extensive battery of motor and action fMRI tasks documented concentric somatotopies for each effector, separated by the CON-linked inter-effector regions. The inter-effector regions lacked movement specificity and co-activated during action planning (coordination of hands and feet), and axial body movement (e.g., abdomen, eyebrows). These results, together with prior work demonstrating stimulation-evoked complex actions and connectivity to internal organs (e.g., adrenal medulla), suggest that M1 is punctuated by an integrative system for implementing whole-body action plans. Thus, two parallel systems intertwine in motor cortex to form an integrate-isolate pattern: effector-specific regions (foot, hand, mouth) for isolating fine motor control, and a mind-body interface (MBI) for the integrative whole-organism coordination of goals, physiology, and body movement.

### Main

Beginning in the 1930s, Penfield and colleagues mapped human M1 with direct cortical stimulation, eliciting movements from about half of sites, mostly of the foot, hand, or mouth. Although representations for specific body parts overlapped substantially, these maps gave rise to the textbook view of M1 organization as a continuous homunculus, from head to toe.

In non-human primates, organizational features inconsistent with the motor homunculus have been described. Structural connectivity studies divided M1 into anterior, gross-motor, “old” M1 (few direct projections to spinal motoneurons), and posterior, fine-motor, “new” M1 (many direct motoneuronal projections). Non-human primate stimulation studies showed the body to be represented in anterior M1, and the motor effectors (tail, foot, hand, mouth) in posterior M1. Such studies also suggested that the limbs are represented in concentric functional zones progressing from the digits at the center, to the shoulders on the periphery. Moreover, stimulations could elicit increasingly complex and multi-effector actions when moving from posterior to anterior M1.

During natural behavior, voluntary movements are part of goal-directed actions, initiated and controlled by executive regions in the CON. Neural activity preceding voluntary movements can first be detected in the dorsal anterior cingulate cortex (dACC) or rostral cingulate zone, then in the pre-supplementary motor area (pre-SMA) and SMA, followed by M1. These regions all project to the spinal cord, with M1 as the main transmitter of motor commands down the corticospinal tract. Efferent motor copies are received by primary somatosensory cortex (S1), cerebellum, and striatum for online correction and learning. Tracer injections in non-human primates demonstrated direct projections from anterior M1/CON to internal organs (e.g. adrenal medulla) for preparatory sympathetic arousal in anticipation of action. Post-movement error and pain signals are relayed primarily to insular and cingulate regions of the CON, which update future action plans.

Resting state functional connectivity (RSFC) fMRI noninvasively maps the brain's functional networks. Precision functional mapping (PFM) studies rely on large amounts of multi-modal data (e.g., RSFC, tasks) to map individual-specific brain organization in greatest possible detail. Early PFM studies identified separate foot, hand, and mouth M1 regions with their respective cerebellar and striatal targets. These foot/hand/mouth motor circuits were characterized by strong within-circuit connectivity and effector specificity in task fMRI. However, these circuits were relatively isolated and did not include functional connections with control networks such as CON that could support the integration of movement with global behavioral goals. A recent study showed that prolonged dominant arm immobilization strengthened functional connectivity between disused M1 and the CON, suggesting that the CON's role may extend beyond abstract action control and into movement coordination.

Here, the latest iteration of PFM was used with higher resolution (2.4 mm) and greater amounts of fMRI (RSFC: 172-1,813 min/participant; task: 353 min/participant), and diffusion data, to map M1 and its connections with highest detail. Results were verified in group-averaged data from the three largest fMRI studies (Human Connectome Project [HCP], Adolescent Brain Cognitive Development [ABCD] Study, UK Biobank [UKB]; total n˜50,000). Furthermore, the findings were placed in cross-species (macaque vs human), developmental (neonate, infant, child, and adult), and clinical (perinatal stroke) contexts using PFM data.

### Two Distinct Functional Systems Alternate in Motor Cortex

### Precision Functional Mapping of Primary Motor Cortex

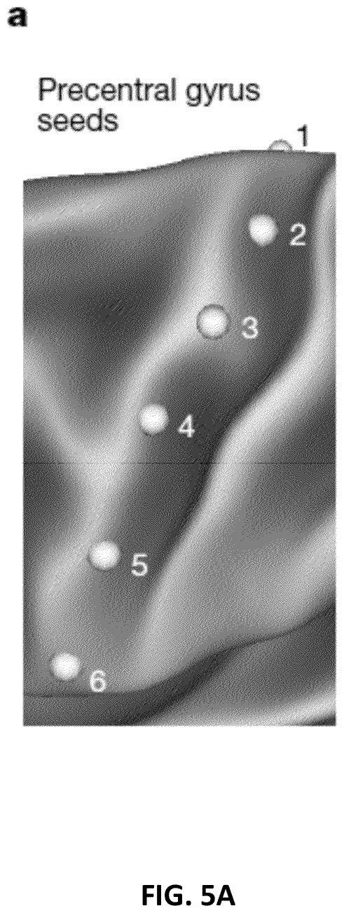

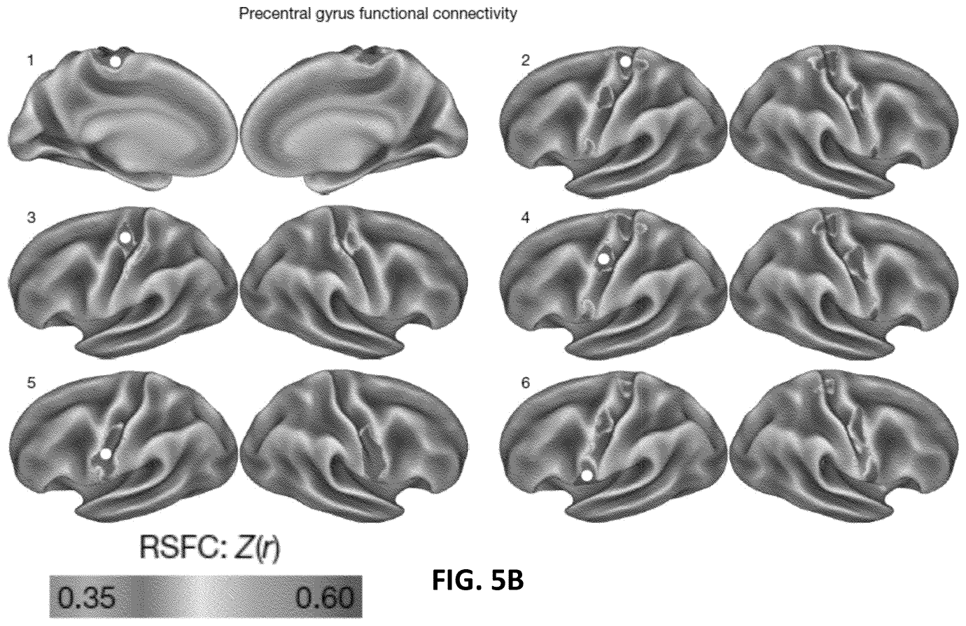

FIGS. 5A and 5B depict resting state functional connectivity (RSFC) seeded from a continuous line of cortical locations in the left precentral gyrus in a single exemplar participant (P1; 356 min resting-state fMRI). The six exemplar seeds shown represent all distinct connectivity patterns observed. Functional connectivity seeded from these locations illustrated classical primary motor cortex connectivity of regions representing the foot (1), hand (3), and mouth (5), as well as an interdigitated set of strongly interconnected regions (2, 4, 6).

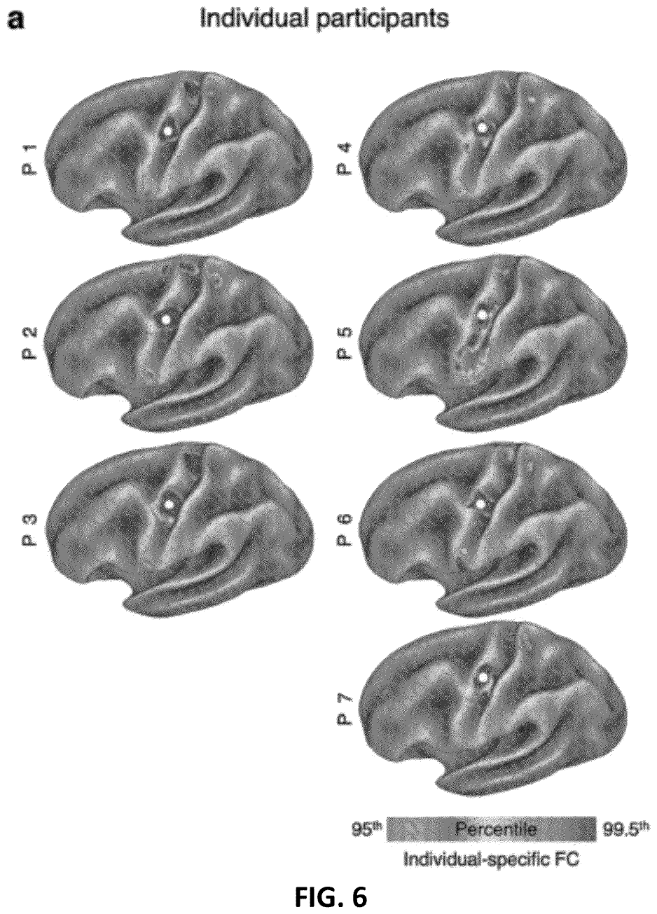

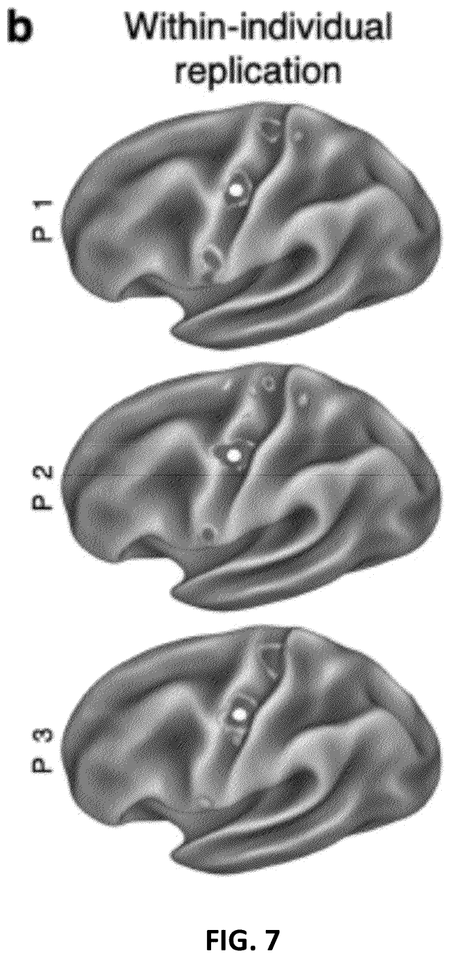

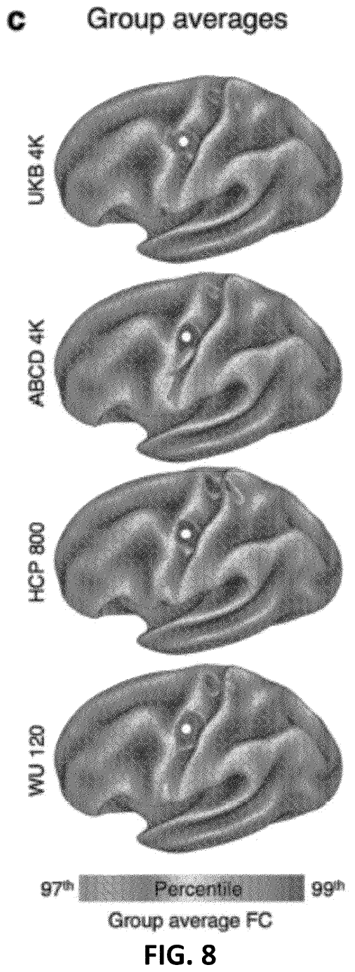

See FIG. 6 for all highly-sampled participants, FIG. 7 for within-participant replications, and FIG. 8 for group-averaged data.

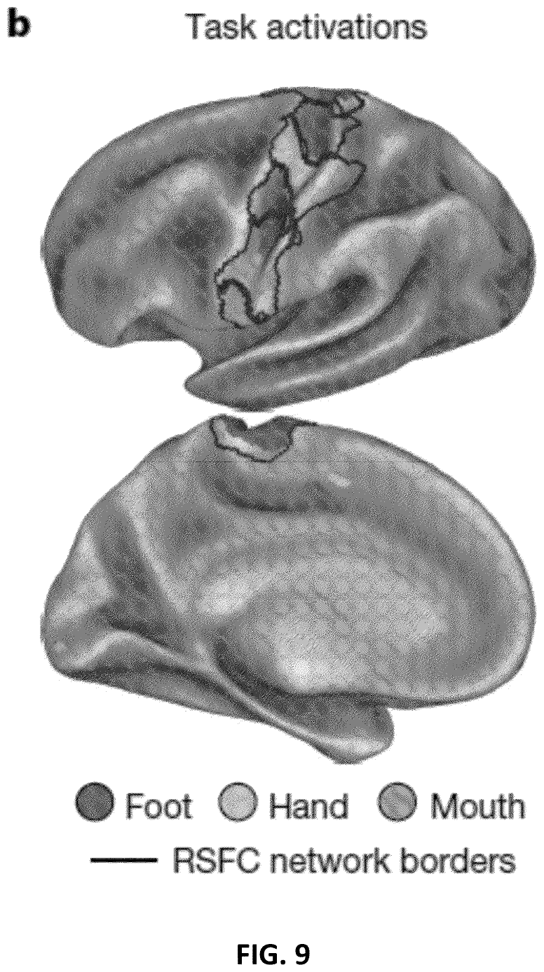

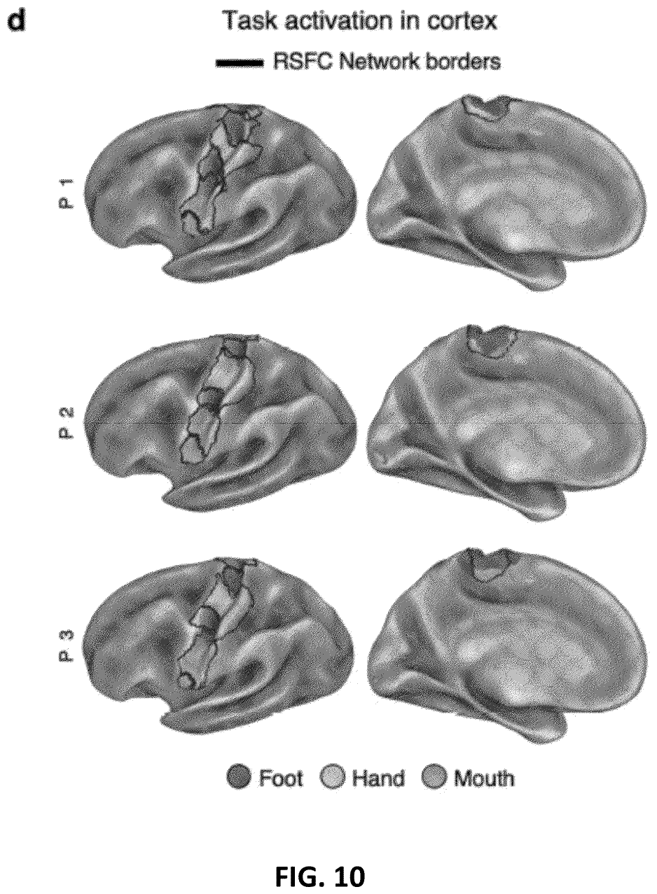

FIG. 9 depicts discrete functional networks that were demarcated using a whole-brain, data-driven, hierarchical approach (see Methods) applied to the resting-state fMRI data, which defined the spatial extent of the networks (black outlines). Regions defined by RSFC were functionally labeled using a classic block-design fMRI motor task involving separate movement of the foot, hand, and tongue. The map illustrates the top 1% of vertices activated by movement of the foot (green), hand (cyan), and mouth (orange) in the exemplar participant (P1; see FIG. 10 for other participants).

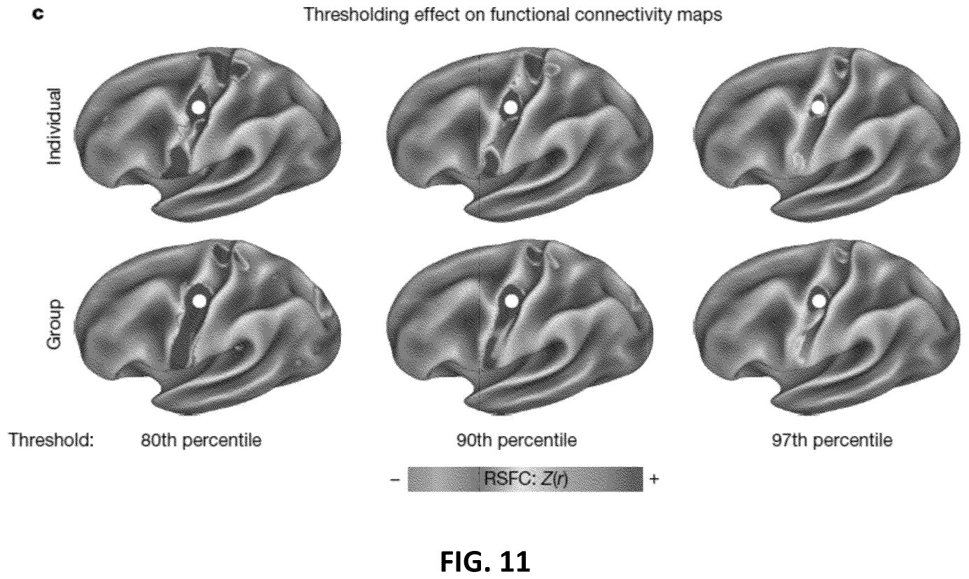

FIG. 11 depicts how the inter-effector connectivity pattern became more distinct from surrounding effector-specific motor regions as connectivity thresholding increased from the 80th to the 97th percentile. RSFC thresholds required to detect the inter-effector pattern were lower in individual-specific data (top) than in group-averaged data (ABCD Study, bottom).

Advanced PFM revealed connectivity that differed strikingly from the canonical homuncular organization of M1. Two contrasting patterns of functional connectivity alternated in primary motor cortex (FIGS. 5A and 5B). The expected pattern, as previously described for M1 foot, hand, and mouth representations, was comprised of three regions (per hemisphere) for which cortical connectivity was restricted to homotopic contralateral M1, SMA and adjacent S1 (FIG. 5B, seeds 1, 3, 5). This set of RSFC-defined regions corresponded with task-evoked activity during foot, hand, and tongue movements (FIG. 9; see FIG. 10 for other participants).

Interleaved between the known foot/hand/mouth M1 regions lay three areas that were strongly functionally connected to each other, both contralaterally and ipsilaterally, forming a previously unrecognized interdigitated chain down the precentral gyrus (FIG. 5B, seeds 2, 4, 6). The motif of three M1 inter-effector regions was observed in every highly-sampled adult (FIG. 6) and replicated within-individual in separate data from the same participants (FIG. 7). Importantly, the inter-effector pattern was also evident in all large-N group-average datasets (UKB, ABCD, HCP, WU120; FIG. 8; see Table 1 for region of interest coordinates).

Table 1: Inter-effector coordinates. Centroid location of inter-effector and effector-specific regions within the HCP data. Coordinates are represented as [X Y Z] in MNI space.

TABLE 1

| | |
|---|---|
| MNI Coordinates | Location |

Inter-effector

| | |
|---|---|
| −18 −27 60 | Left dorsal M1 |
| −38 −14 46 | Left middle M1 |
| −59 3 14 | Left ventral M1 |
| 20 −26 60 | Right dorsal M1 |
| 42 −11 47 | Right middle M1 |
| 60 3 13 | Right ventral M1 |
| −28 −12 2 | Left posterior putamen |
| 30 −11 2 | Right posterior putamen |
| −15 −17 −2 | Left Ventral Intermediate nucleus of thalamus |
| 16 −17 −2 | Right Ventral Intermediate nucleus of thalamus |
| 0 −69 −21 | Cerebellar vermis |

Foot

| | |
|---|---|
| −4 −27 70 | Left medial M1 |
| 5 −23 72 | Right medial M1 |
| −11 −22 2 | Left Centromedian nucleus of thalamus |
| 12 −21 3 | Right Centromedian nucleus of thalamus |
| −16 −36 −20 | Left anterior cerebellum |
| 14 −34 −22 | Right anterior cerebellum |

Hand

| | |
|---|---|
| −36 −24 60 | Left dorsal M1 |
| 40 −21 59 | Right dorsal M1 |
| −20 −48 −18 | Left anterior cerebellum |
| 16 −48 −18 | Right anterior cerebellum |

Face

| | |
|---|---|
| −49 −8 29 | Left ventral M1 |
| 50 −5 28 | Right ventral M1 |
| −26 −10 0 | Left posterior putamen |
| 24 −6 −2 | Right posterior putamen |
| −14 −18 0 | Left Ventral Intermediate nucleus of thalamus |
| 12 −16 2 | Right Ventral Intermediate nucleus of thalamus |
| −16 −58 −18 | Left anterior cerebellum |
| 14 −58 −18 | Right anterior cerebellum |

The M1 inter-effector functional connectivity motif was most apparent in individual-specific maps, but once recognized, was also clearly identifiable in group-averaged data when visualized using stringent connectivity thresholds (FIG. 11).

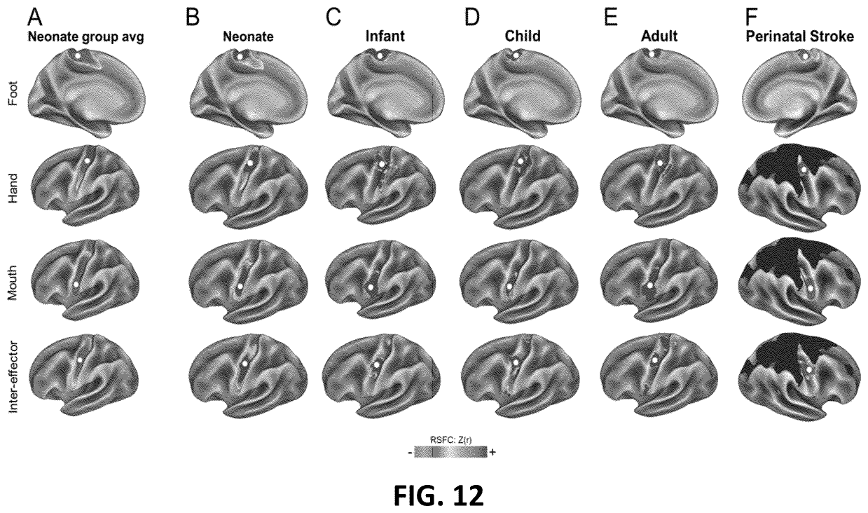

The inter-effector regions were evident relatively early in development. While PFM data from a newborn failed to reveal the inter-effector motif, it was detectable in an 11-month-old infant, and was almost adult-like in a 9-year-old child (FIG. 12A-12E). Inter-effector regions could even be identified in an individual with preserved motor function despite suffering severe bilateral perinatal strokes that destroyed large portions of M1 (FIG. 12F).

### M1 Functional Connectivity in Pediatric Participants and a Perinatal Stroke Participant

Functional connectivity maps were seeded from a continuous line of points down precentral gyrus in fMRI data from data averaged across 262 human neonates, all scanned shortly after birth (FIG. 12A); a neonate scanned 13 days after birth (FIG. 12B); an 11-month old infant (FIG. 12C); a 9-year old child (FIG. 12D); adult participant P01 (FIG. 12E, from FIGS. 6 and 7); and an adolescent who had experienced extensive cortical reorganization after severe bilateral perinatal strokes (FIG. 12F, destroyed cortex in black). Right hemisphere is shown in the stroke patient because left hemisphere M1 was entirely lost. Example seed maps shown here illustrate observed effector-specific (first three rows) and inter-effector (fourth row) connectivity. Effector-specific and inter-effector regions exhibited clear boundaries within M1 in the infant, child, the adults, and the stroke patient, but not in the neonates. Visualization thresholds varied between 0.3 and 0.5 across datasets due to differences in data collection and processing, as well as differences inherent to the populations.

### Inter-Effector Regions are Functionally Connected to Cingulo-Opercular Control Network

### Functional Connectivity and Cortical Thickness of the Motor Cortex Inter-Effector Motif

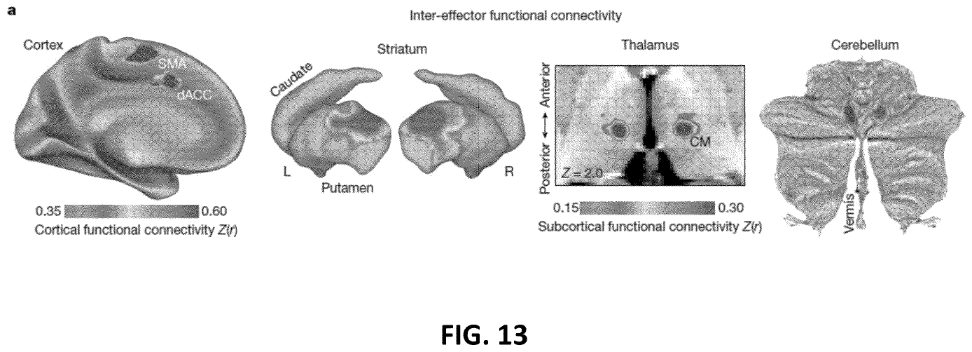

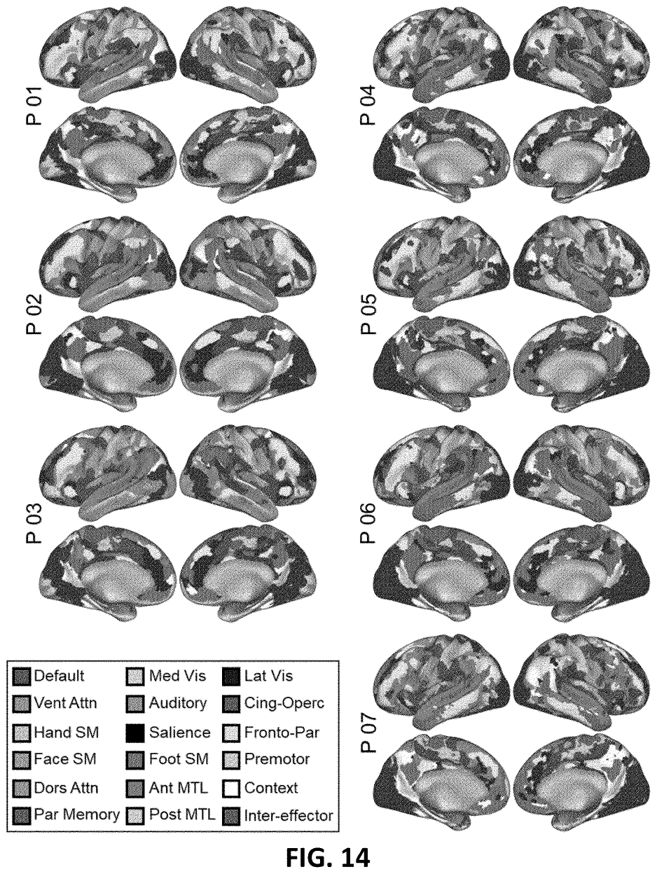

FIG. 13 depicts brain regions with the strongest functional connectivity to the left middle inter-effector region (exemplar seed) in cortex, striatum, thalamus (horizontal slice; centromedian (CM) nucleus shown), and cerebellum (flat map) in the exemplar participant (P1); See FIG. 14 for other participants.

Discrete functional networks were demarcated within each subject in M1 and S1 using a whole-brain, data-driven hierarchical community detection approach applied to the resting-state fMRI data. Communities were matched to known large-scale networks, and to the inter-effector regions. Networked inter-effector regions were observed in all participants.

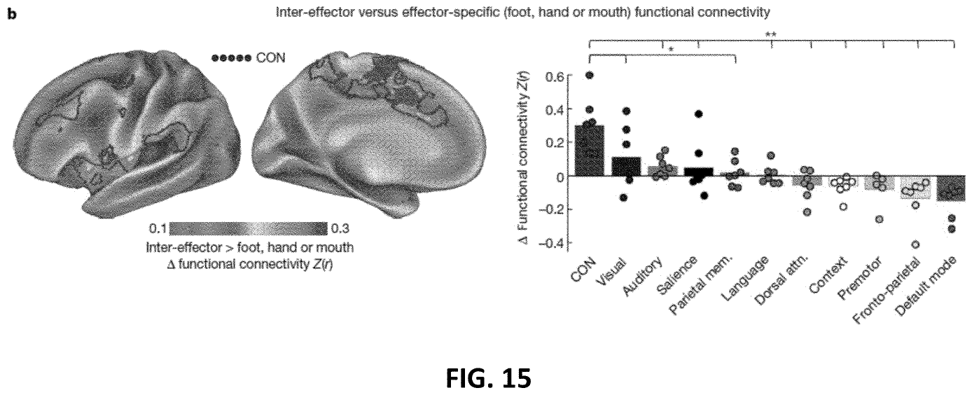

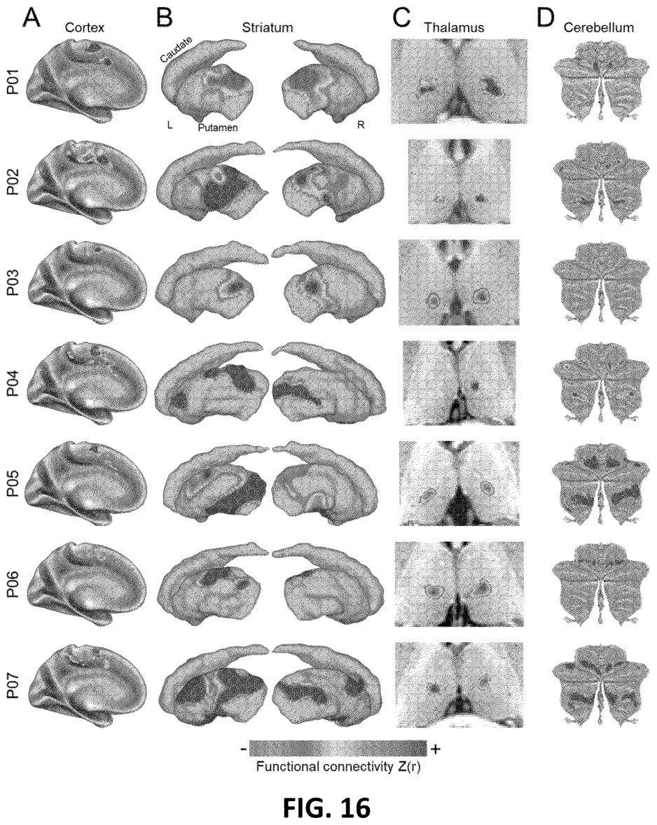

FIG. 15 depicts brain regions more strongly functionally connected to inter-effectors than to any foot/hand/mouth regions (P1; FIG. 16A) for other participants). Purple outlines show the Cingulo-opercular Network (CON; individual-specific). Central sulcus is masked as it exhibits large differences by definition.

### Whole Brain Functional Connectivity of Precentral Gyrus Inter-Effector Motif Across Subjects

Brain regions with the strongest functional connectivity to the middle inter-effector region in medial cortex (FIG. 16A), striatum (FIG. 16B, lateral view of left and right striatum), thalamus (FIG. 16C, axial view), and cerebellum (FIG. 16D). Functional connectivity values are thresholded at Z(r)>0.35 in cortex. Subcortical functional connectivity values are thresholded at different levels in each subject due to variation in subcortical signal-to-noise ratios across individuals. Thresholds were chosen to illustrate the strongest subcortical connections. Specific thresholds shown here: P01—Z(r)>0.15; P03, 04, 06, 07—Z(r)>0.10; P02—Z(r)>0.04; P05—Z(r)>0.03. Connectivity was calculated between every network and both inter-effector and effector-specific M1 regions. The plot shows the smallest difference between inter-effector connectivity and any effector-specific connectivity (standard error bars). This difference was larger for CON than for any other network (*: P<0.05).

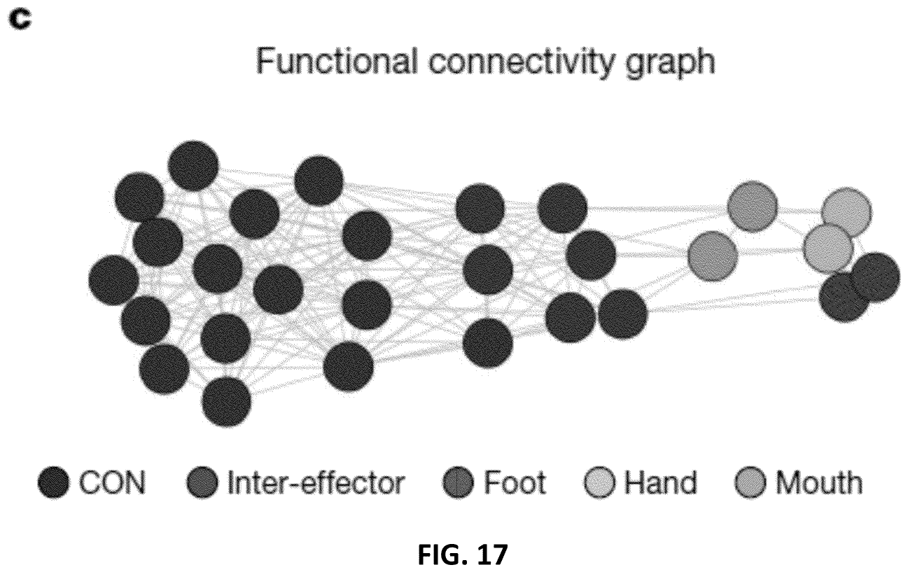

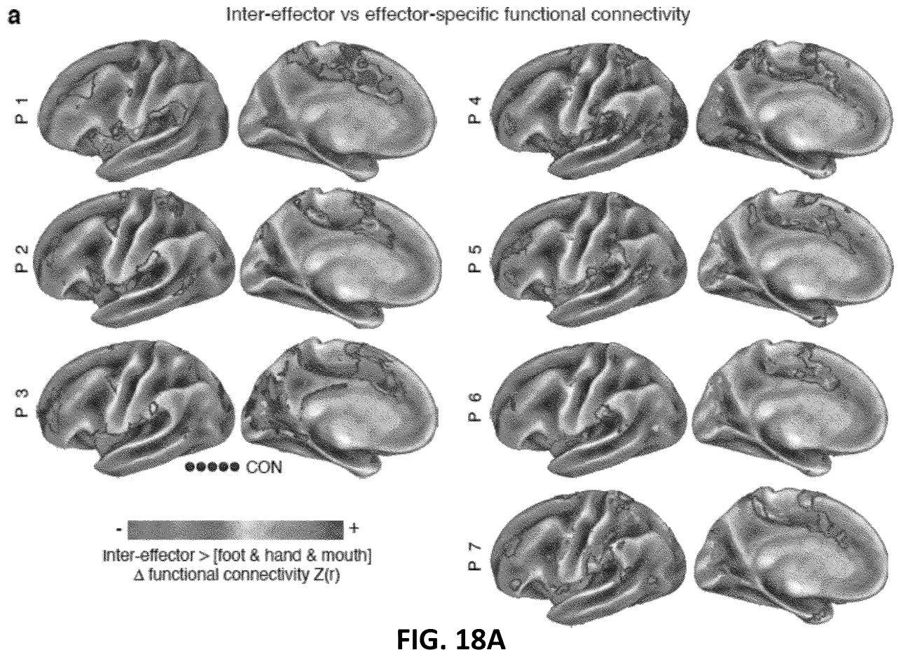

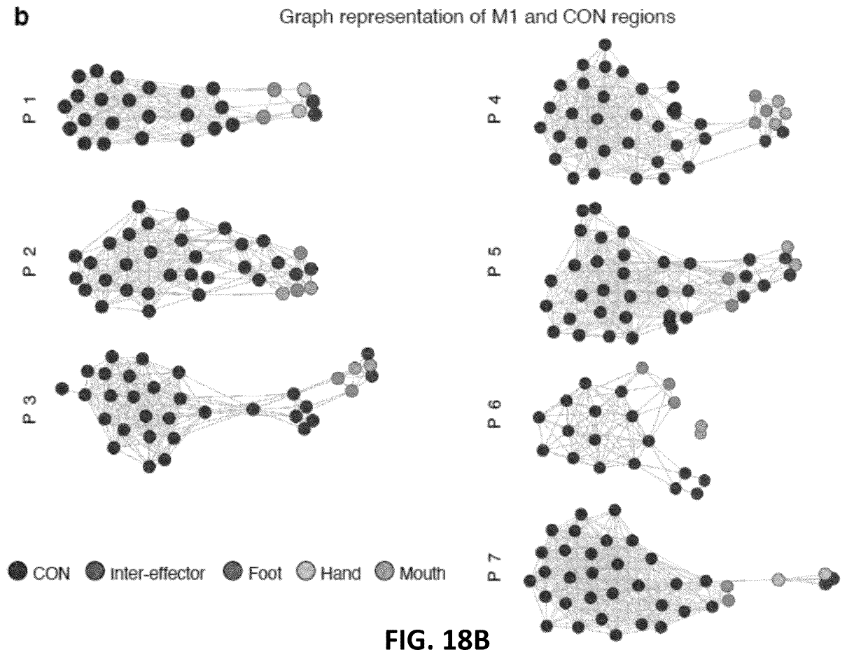

Referring to FIG. 17, inter-network relationships visualized in network space using a spring-embedding plot, in which connected regions are pulled together while disconnected regions are pushed apart. Connecting lines indicate a functional connection (r>0.2) (P1; FIG. 18B for all participants).

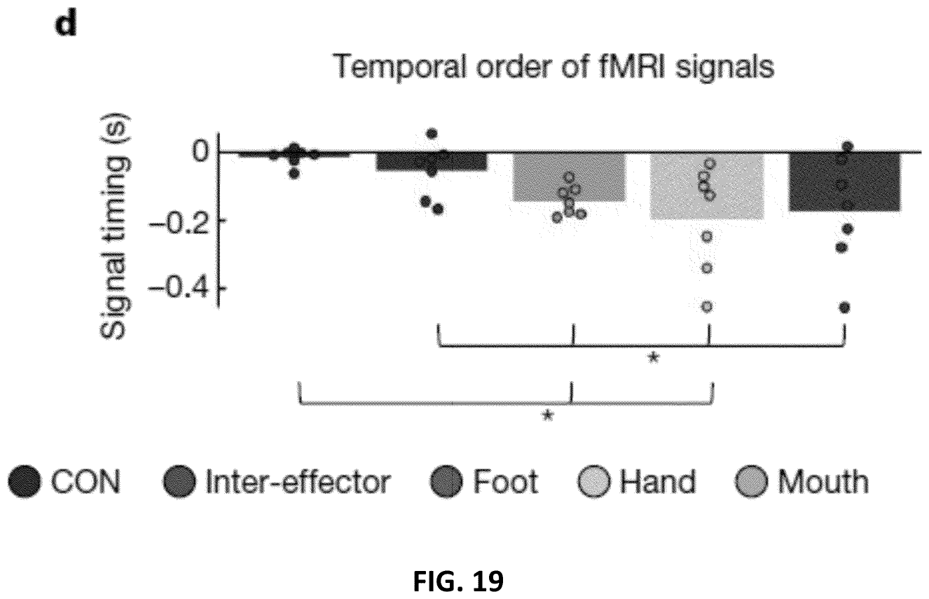

FIG. 19 depicts inter-effector and effector-specific regions tested for systematic differences in the temporal ordering of their infra-slow fMRI signals (<0.1 Hz). Plot shows across-participant average signal ordering in CON, inter-effector, and effector-specific regions (standard error bars; *: P<0.05). Prior electrophysiology work suggests that later infra-slow activity (here, CON) corresponds to earlier delta-band (0.5-4 Hz) activity.

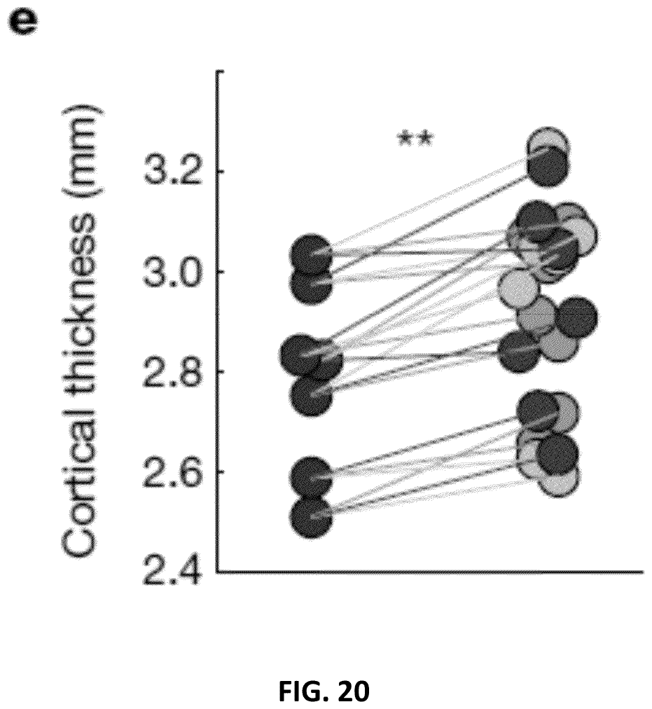

Referring to FIG. 20, in each participant (individual dots), inter-effector regions exhibited lower cortical thickness than all effector-specific regions (**: P<0.01).

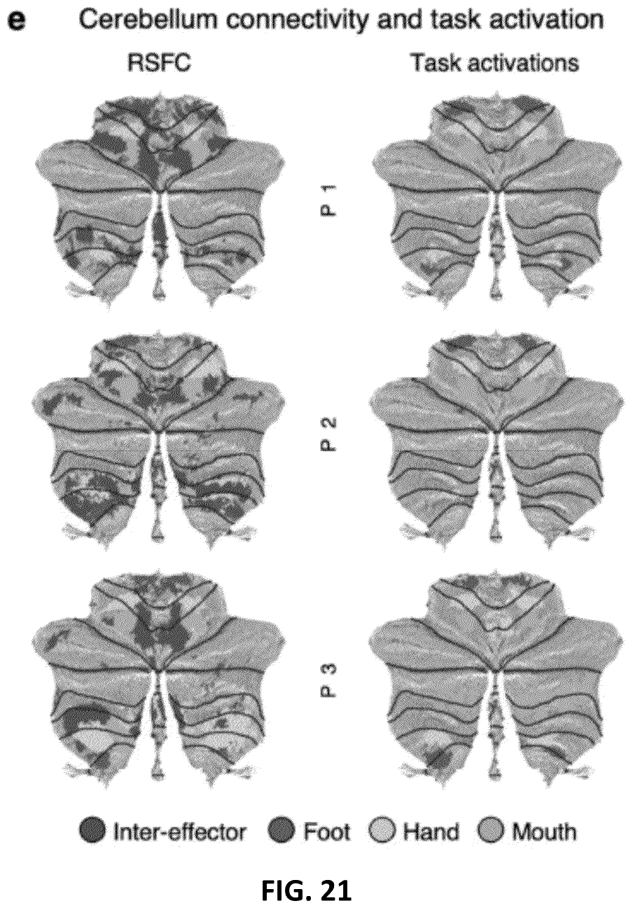

In addition to being interconnected, the three inter-effector regions were functionally connected to the dACC and pre-SMA, thought to be important for goal-oriented cognitive control. In subcortex, inter-effector regions were most strongly connected to dorsolateral putamen, and to ventral intermediate (VIM), centromedian (CM), ventral posteriomedial (VPM), and ventral posterior inferior (VPI) nuclei of the thalamus (FIG. 13; see FIG. 14 for other participants). Inter-effector regions were strongly connected to cerebellar areas surrounding but distinct from effector-specific cerebellar regions (FIG. 21).

In all highly-sampled individuals (n=7), the inter-effector regions had stronger connections to CON than did any of the foot/hand/mouth regions (FIG. 15; FIG. 16 for all participants; across participants: all paired t>4.75; all P<0.01; FIG. 18A). The inter-effector vs. foot/hand/mouth difference was larger for CON than for any other network (all paired t>2.8; all P<0.03; FIG. 15). In network space, inter-effector regions were positioned between CON and the foot/hand/mouth regions (FIG. 17; FIG. 18B for all participants). Inter-effector regions were also more strongly connected to: middle insula, known to process pain and interoceptive signals (FIG. 18B; all paired t>2.7; all P<0.03); lateral cerebellar lobule V and vermis Crus II, lobule VIIb, and lobule VIIIa (all paired t>3.7, all P<0.01); dorsolateral putamen, critical for motor function (all paired t>3.7; all P<0.01); and sensory-motor regions of thalamus (VIM; CM; VPM; all paired t>3.0, all P<0.02).

Comparing the relative timing of resting-state fMRI signals (lag structure) showed that infra-slow (<0.1 Hz) fMRI signals in both the CON and the inter-effector network lagged behind those in effector-specific regions (FIG. 19; CON vs foot: paired t=2.38, P=0.055; vs hand and mouth: all t>2.84, all P<0.03; inter-effector vs foot/hand/mouth: all t>2.5, all P<0.05). Inter-regional lags in infra-slow (<0.1 Hz) signals are associated with propagation of higher-frequency delta activity (0.5-4 Hz) in the opposite direction, suggesting that high-frequency signals may occur earlier in CON than M1—consistent with electrical recordings during voluntary movement—but that such signals reach the inter-effectors earlier than the foot, hand, mouth regions.

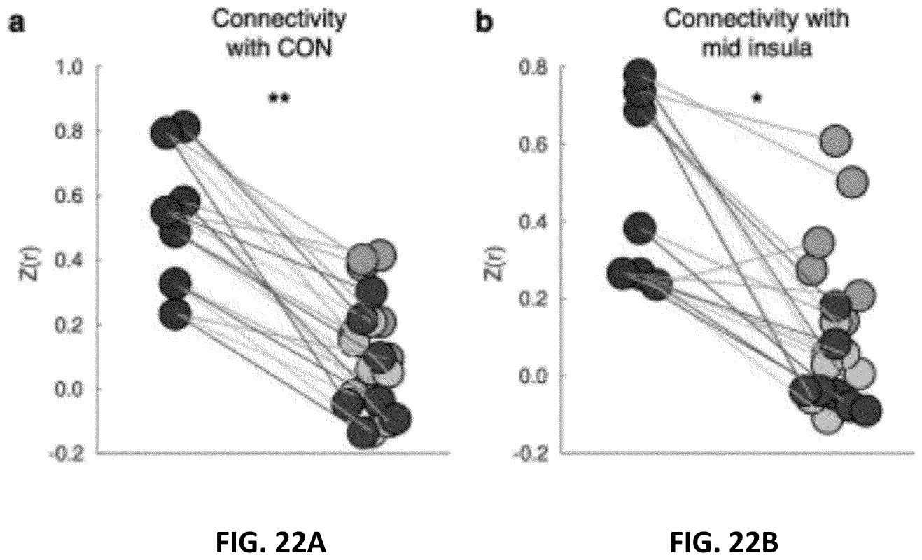

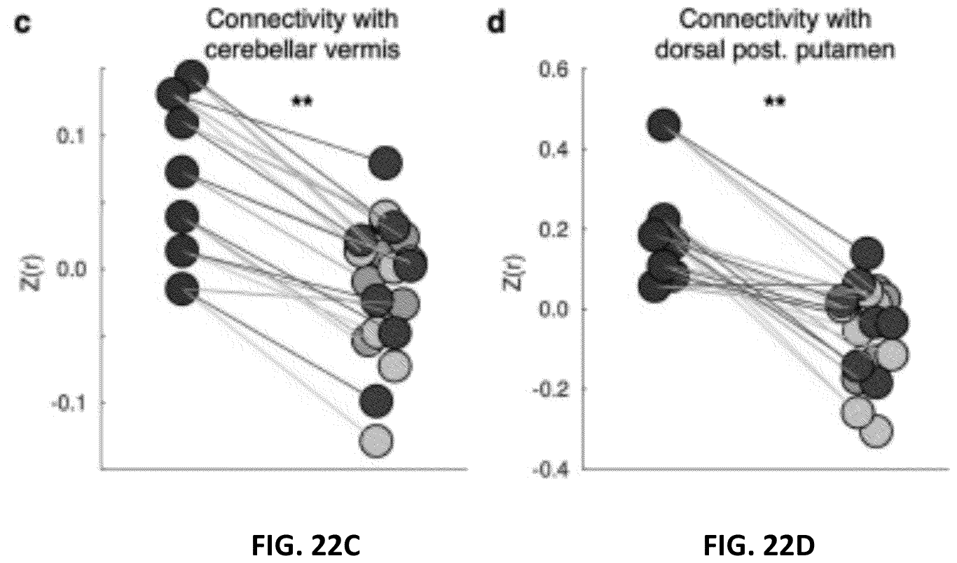

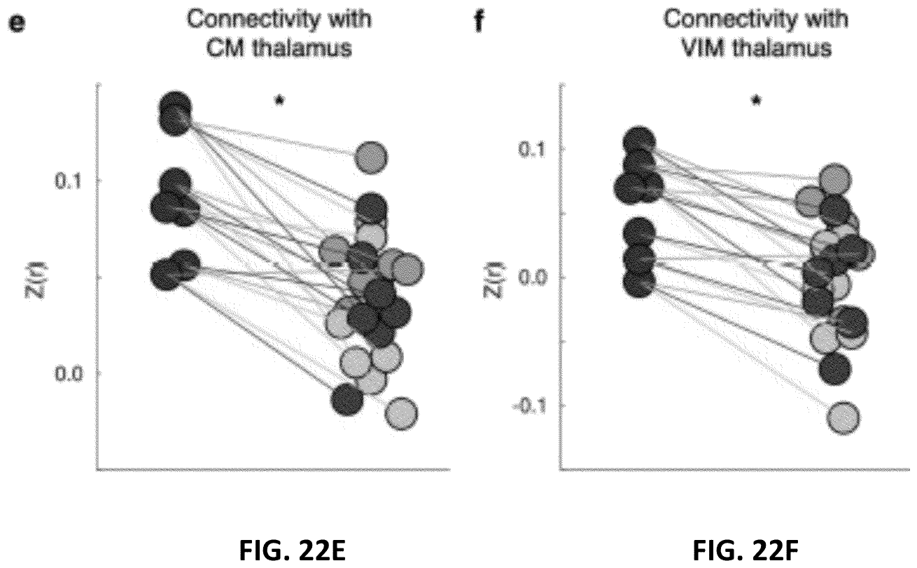

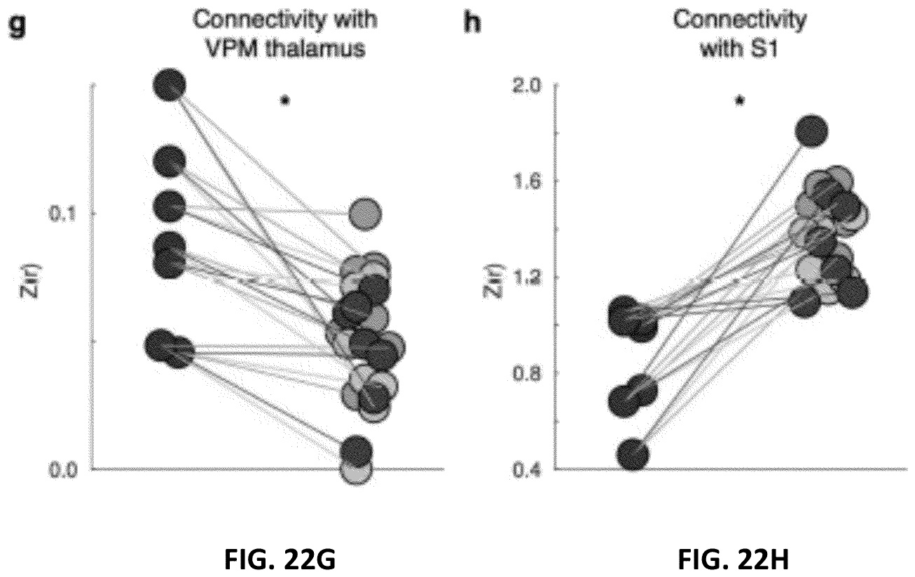

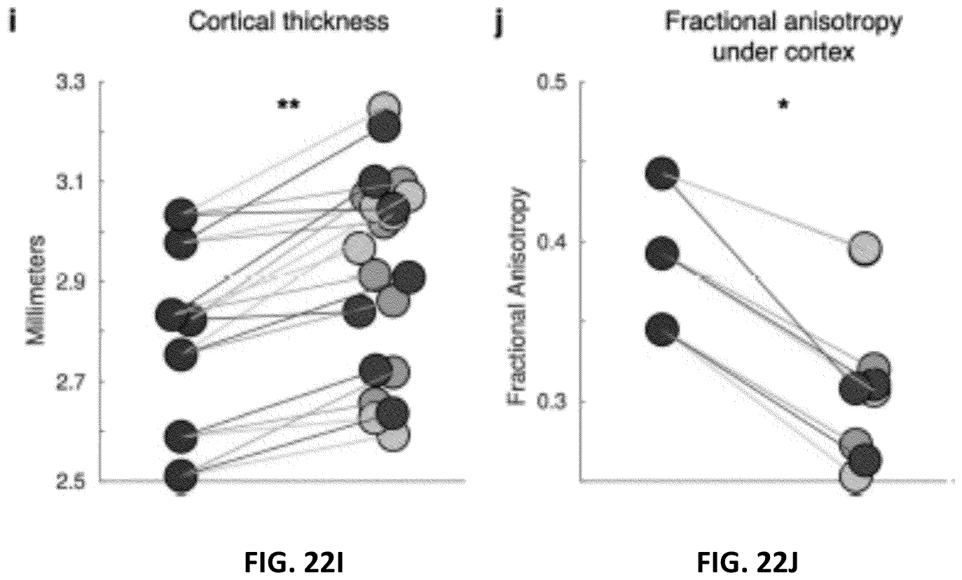

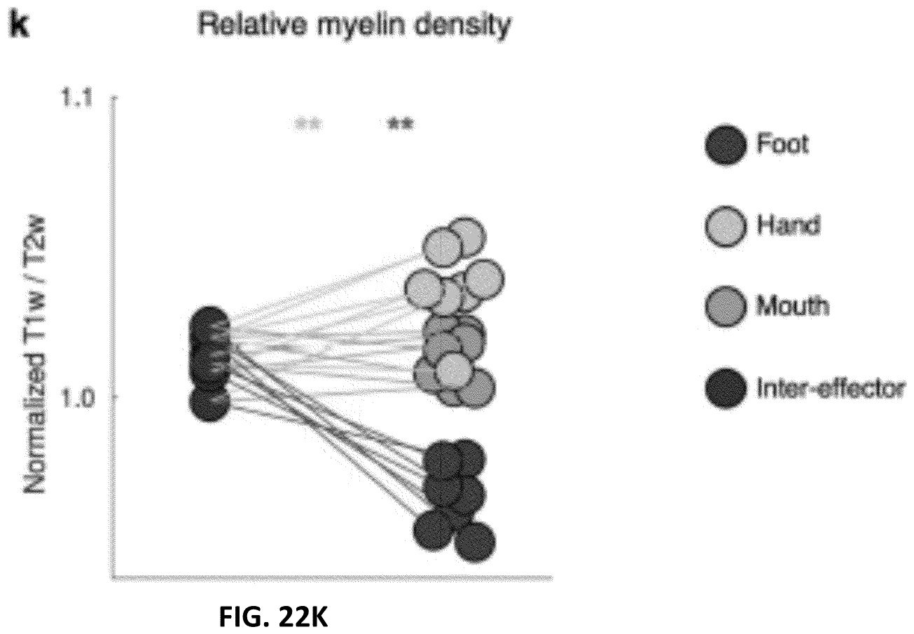

As expected, the M1 foot/hand/mouth regions were strongly functionally connected with adjacent S1 (FIGS. 5A and 5B, FIG. 22A), consistent with known functional connections between M1 and S1. By contrast, inter-effector regions exhibited lower connectivity with adjacent S1 (FIG. 22H; all paired t>3.2, all P<0.02). More specifically, inter-effector functional connectivity extended into the fundus of the central sulcus (FIG. 22B; Brodmann Area [BA]3a), which represents proprioception, but not to the postcentral gyrus (BAs 3b/1/2) representing cutaneous tactile stimuli.

Convergent with these functional differences, metrics of brain structure systematically differed between inter-effector and effector-specific regions. Inter-effector regions exhibited lower cortical thickness (all paired t>3.6; all P<0.01; FIG. 20), more similar to prefrontal cortex, but higher fractional anisotropy (2 mm beneath cortex; all paired t>5.3; all P<0.05; FIG. 22J). Inter-effector regions also had higher intracortical myelin content than foot regions (paired t=6.8, P<0.001) but lower than hand regions (paired t=4.8, P=0.003; FIG. 22K).

### Functional Connectivity and Structural MRI Metrics of Precentral Gyrus Regions

In each individual participant, measures derived from each of the foot, hand, mouth, and inter-effector motor regions. Colored lines connect the same participant's inter-effector and effector-specific regions for ease of comparison. Functional connectivity strength between M1 region and individual-specific cingulo-opercular network (FIG. 22A); Functional connectivity strength between M1 region and middle insula (FIG. 22B); Functional connectivity strength with Lobule VIIIa vermis of the cerebellum (FIG. 22C); Functional connectivity strength between M1 region and dorsal posterior putamen (FIG. 22D). Functional connectivity strength between M1 region and nuclei of the thalamus: Ventral Intermediate nucleus (FIG. 22E); Parafascicular nucleus (FIG. 22F); (FIG. 22G) Ventral Lateral Anterior nucleus. Functional connectivity strength between M1 region and adjacent postcentral gyrus (FIG. 22H). Cortical thickness in M1 region (FIG. 22I). Fractional Anisotropy within 2 mm below cortex under M1 region (FIG. 22J). Intracortical myelin, indexed by the T1/T2 ratio and normalized across cortex (FIG. 22K), within cortex of M1 region. * p<0.05; ** p<0.01; *** p<0.001.

### Concentric Effector Somatotopies are Separated by Inter-Effector Body/Action Regions

### Individual-Specific Task Activations in Primary Motor Cortex

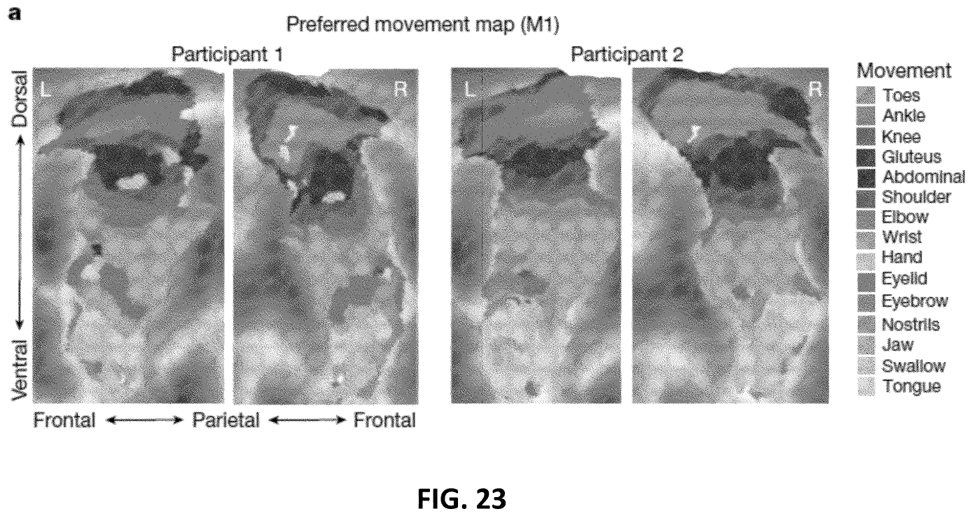

FIG. 23 depicts task fMRI activations (P1, P2) during a movement task battery, including movement of the toes, ankles, knees, gluteus, abdominals, shoulders, elbows, hands, eyebrows, eyelids, tongue, and swallowing (244 min/participant). Each cortical vertex is colored according to the movement that elicited the strongest task activation (winner-take-all) and is shown on a flattened representation of the cortical surface. Background shading indicates sulcal depths.

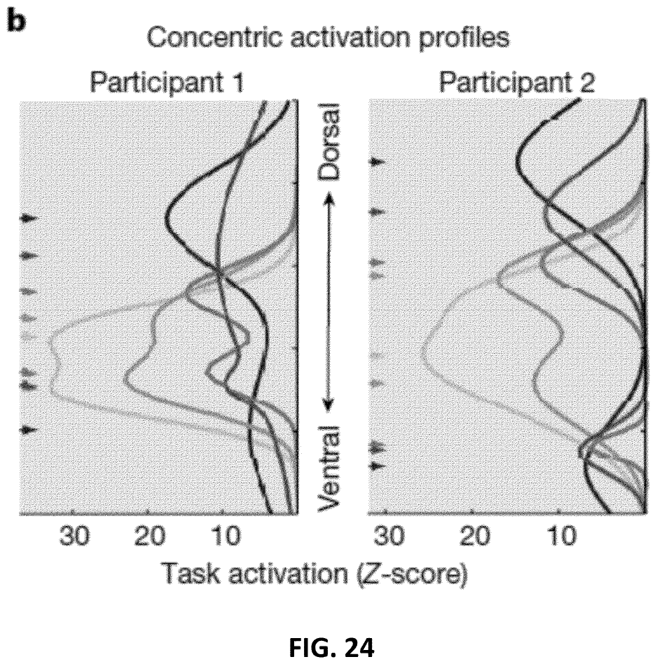

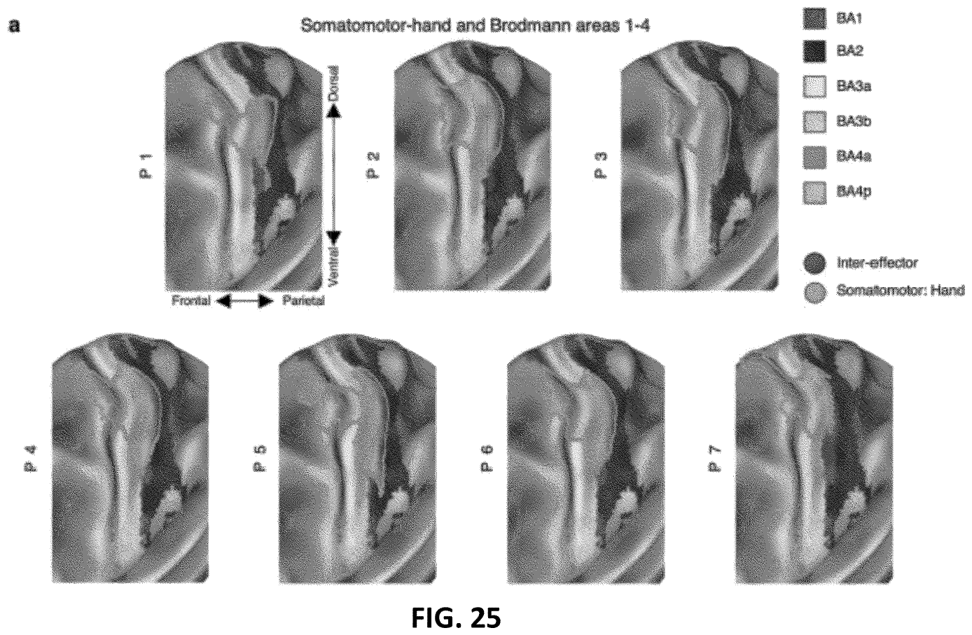

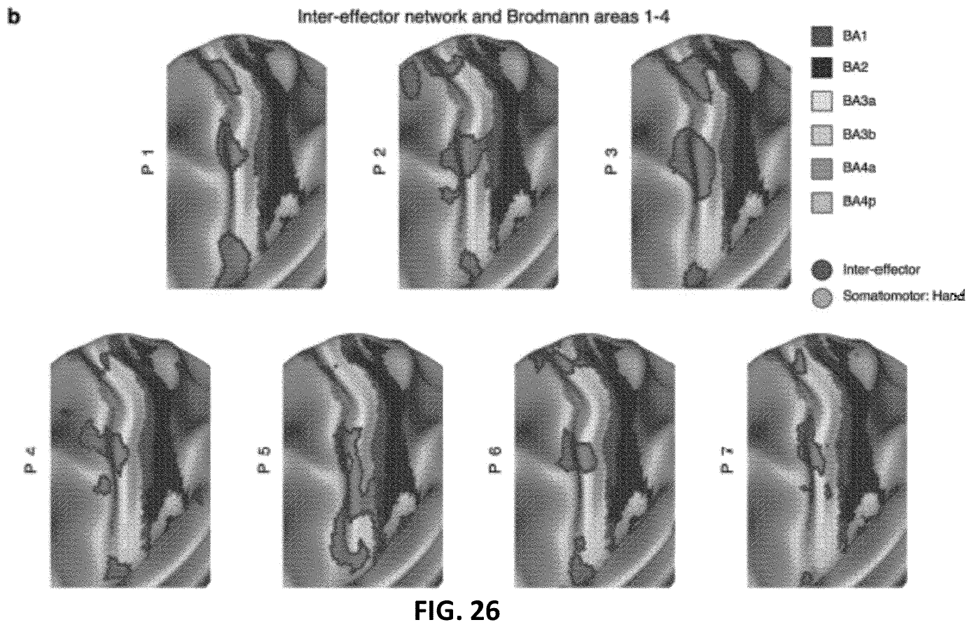

FIG. 24 depicts activation strength for each movement was computed along the dorsal-ventral axis within M1. A two-peak gaussian curve was fitted to each movement activation (see Methods). Fitted curves are shown for movement of abdominals, shoulder, elbow, wrist, and hand. Peak locations (hashes on right) were arranged concentrically around the hand peak. See FIGS. 25 and 26 for all movements.

### Inter-Effector and Effector-Specific Regions in Pre- and Postcentral Gyrus

In every participant, Brodmann areas in M1 (BAs 4a, 4p) and S1 (BAs 1, 2, 3a, 3b) are displayed on the cerebral cortex, tilted around the Y- and Z-axes to show S1. Overlaid are the somatomotor-hand region (FIG. 25), and the inter-effector regions (FIG. 26).

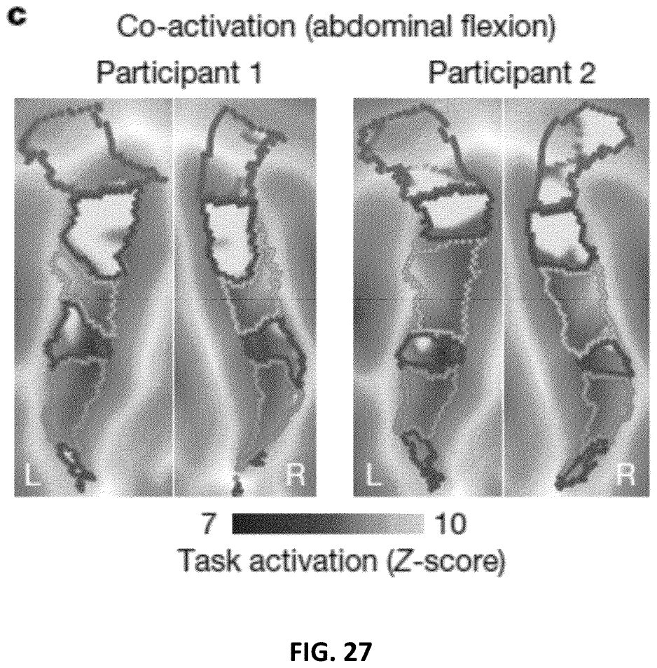

In FIG. 27, inter-effector regions were co-activated during abdominal contraction.

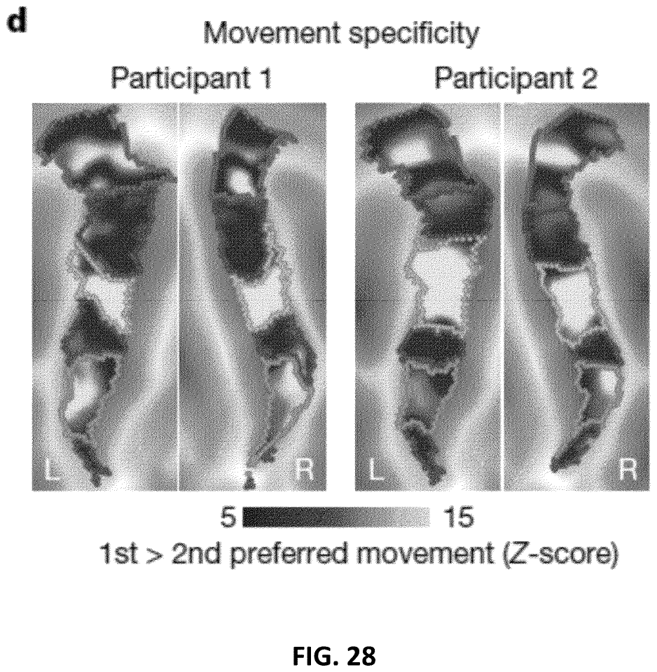

In FIG. 28, inter-effector regions exhibited more generalized evoked activity during movements. Movement specificity was computed as the activation difference between the first and second-most preferred movements for the six conditions that most activated each discrete region (toes, abdominal, hand, eyelid, tongue, swallowing).

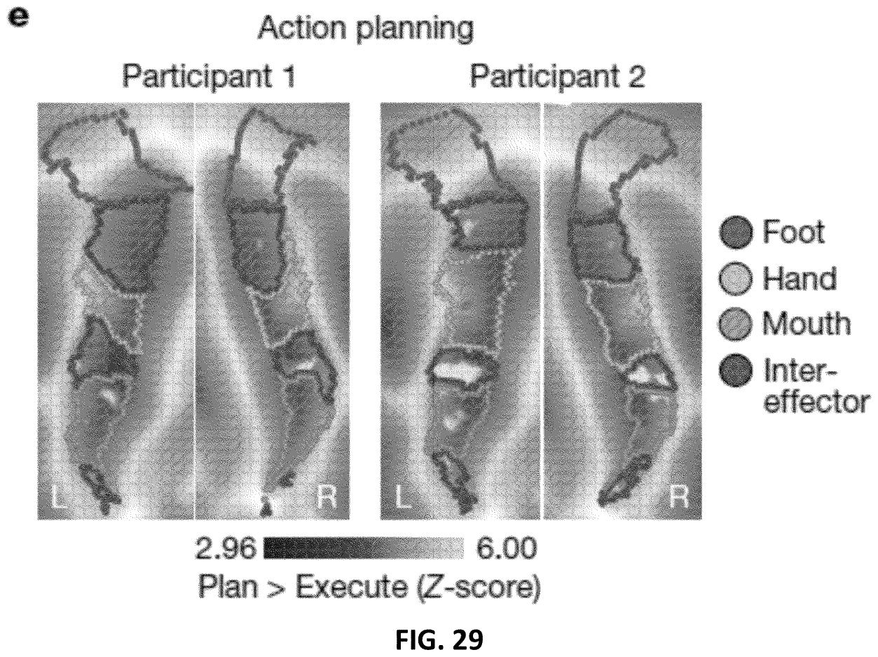

FIG. 29 depicts event-related task fMRI data during an action planning task with separate planning and execution phases for movements of the hands and feet (see Methods). M1 activity in the planning phase was higher than in the execution phase in the inter-effector but not the effector-specific regions.

To better understand the functions of the inter-effector motif, fMRI data was collected during blocked performance of twenty-five different movements in two highly-sampled individuals (64 runs; 244 min/participant) and during a novel event-related task with separate planning and execution phases for coordinated hand and foot movements (12 runs; 132 min/participant). According to the homuncular model of M1, activation when moving a given body part should exhibit a single peak within the precentral gyrus. If M1 is instead organized into concentric functional zones, all movements except those at the centers (i.e., toes, fingers, tongue) should exhibit two peaks (above and below). Within each of the three effector-specific regions, the topography of preferred movements—the movement eliciting greatest activation in each vertex (FIG. 23)—was more consistent with a concentric organization (distal-proximal; e.g.: toes in the center, with surrounding concentric zones of ankle-knee-hip) than with the canonical, linear toes-to-face homuncular model.

To formally test for a concentric organization, one- and two-peak gaussian curves were fit to the task activation profiles along the dorsomedial-to-ventrolateral axis of M1. Two-peak curve fits were significantly better for all movements (F-test for comparing models: all F>6.9, all P<0.001) except hand in P2 (F≅0, P≅1) (FIG. 25). The curve fits revealed concentric activation zones centered around activation peaks for distal movements (hand (FIG. 24), toes, and tongue (FIG. 26)) and expanding outward to more proximal movements (shoulder, gluteus, jaw). Concentric rings of activation from separate foot/hand/mouth centers intersected in the top and middle inter-effector regions.

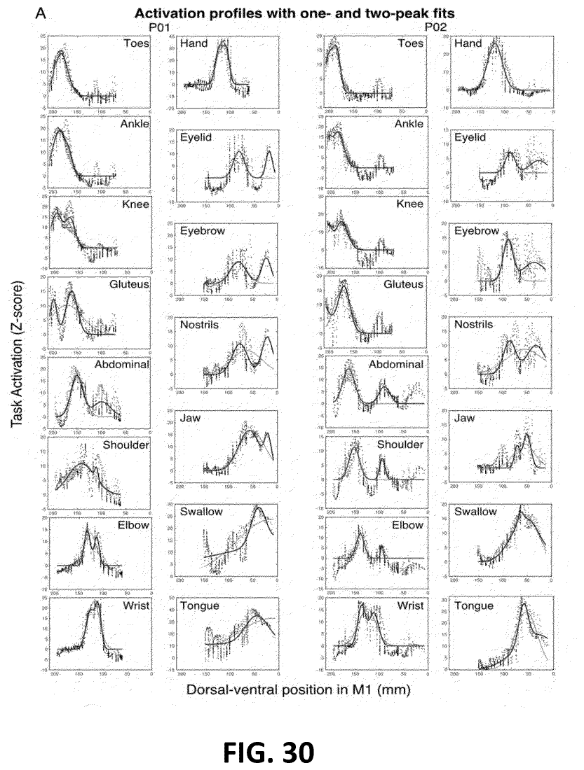

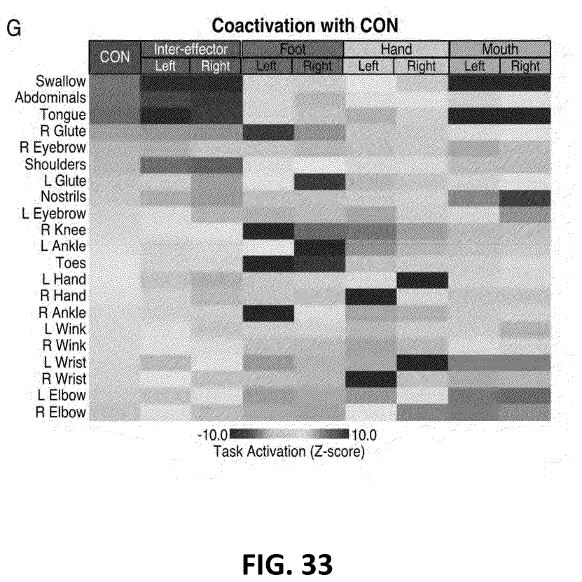

Some movements requiring less fine motor control, such as isometric contraction of the abdominals (FIG. 27), or raising the eyebrow, co-activated multiple inter-effector regions and the CON (FIG. 30-33). In contrast, movements of the foot and hand only activated the corresponding effector-specific regions (FIG. 30-33 Unlike effector-specific regions, the inter-effectors exhibited only weak movement-specificity, with minimal activation differences between their 1st and 2nd most preferred movements (FIG. 28).

FIG. 30 depicts movement-driven activation plotted against dorsal-ventral position within left hemisphere M1 for all movement tasks. For most movements, a two-peak curve (blue), modeled as a double-gaussian, fit the data better than a one-peak curve (red), modeled as a single-gaussian (ps<0.001).

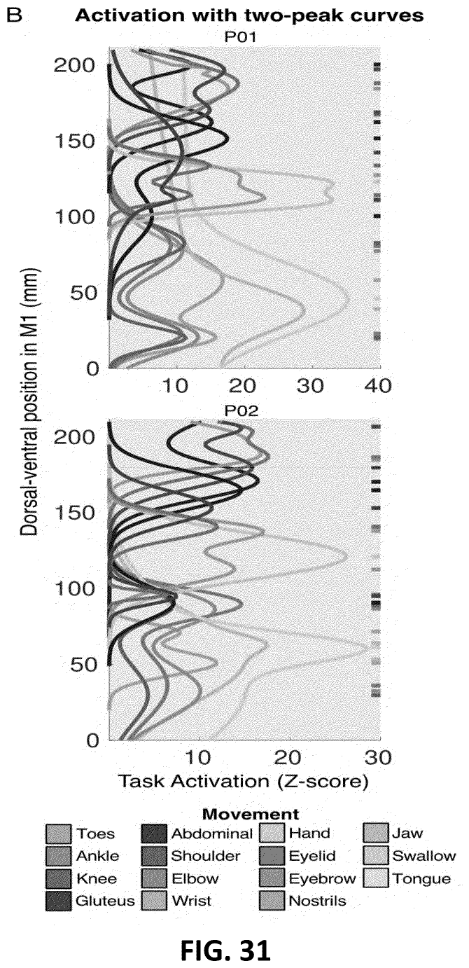

FIG. 31 depicts all modeled two-peak curves for P01 and P02. Most movements, except those by the distal-most body parts (toes, hand, tongue), showed a clear double peak. Peak locations (hashes on right) were arranged concentrically around the peaks of the distal-most body parts.

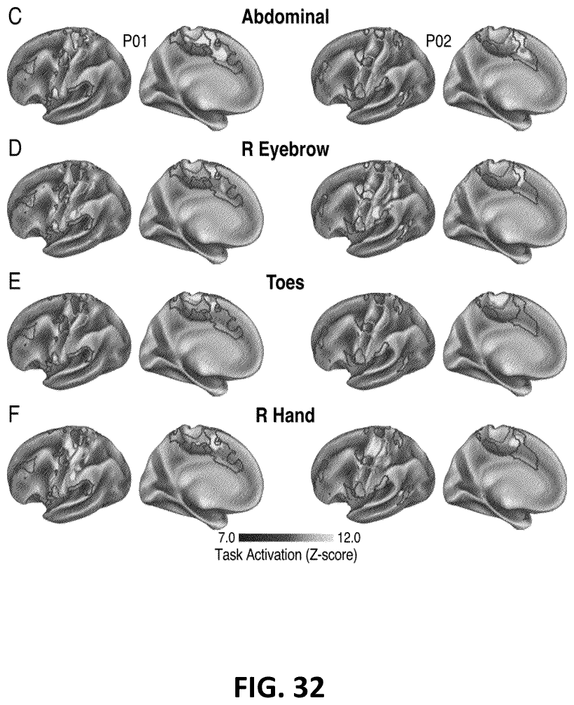

FIG. 32 depicts that each participant, in the abdominal flexure task and the eyebrow raising task, the inter-effector regions and CON were active. By contrast, in toe and hand motion tasks, activation was much more specific to a single region of somatomotor cortex. Across tasks, the degree of CON activation was consistently similar to the activation of the inter-effector regions (correlation between CON and inter-effector activations: all rs>0.81, ps<10-5), but not consistently to hand (CON vs hand: rs>0.05, ps<0.82) or foot (CON vs foot: rs>0.33, ps<0.13) regions, and more weakly to mouth regions (CON vs mouth: rs>0.61, ps<0.003). Illustrated activation values are averaged across participants and ordered based on CON activation.

Regions in CON instantiate action plans, suggesting the CON-to-inter-effector connection could carry general action planning signals. Across foot and hand movements in the novel coordination task, the inter-effectors showed greater activity during action planning than movement execution, but the effector-specific regions did not (FIG. 29), suggesting that the implementation of action plans may be enabled in part by the inter-effector regions in M1.

### Precentral Gyrus Functional Connectivity in Non-Human Primates

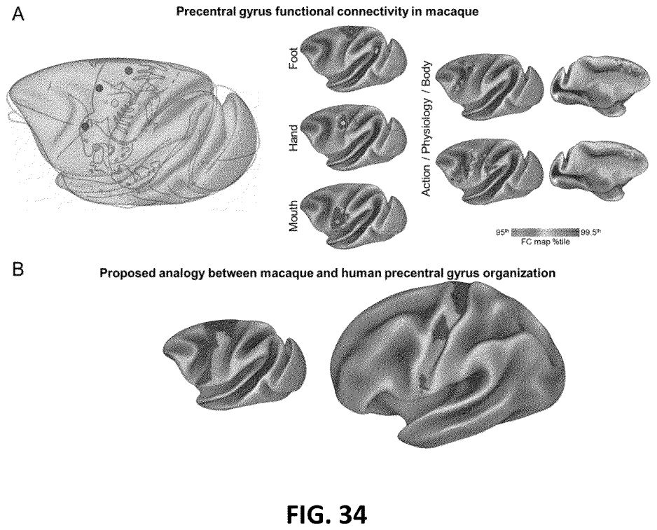

FIG. 34A depicts functional connectivity maps that were seeded from points in precentral gyrus in a macaque. Left: seed locations are shown relative to the macaque “simiculus” (Woolsey et al., 1952). Middle: maps seeded from posterior precentral gyrus locations corresponding to the foot (green), hand (blue), and mouth (orange) exhibited clear boundaries within M1 in the macaque, but no pattern of distributed connectivity interdigitated could be detected between the foot, hand, and mouth regions. Right: seeds in the anterior portion of precentral gyrus (maroon) exhibited strong connectivity distributed along a dorsal-ventral axis within anterior precentral gyrus, as well as with homologues of human CON regions, including inferior frontal gyrus, anterior inferior parietal cortex, and anterior dorsomedial prefrontal cortex. These anterior seeds corresponded to the body and neck, as well as to regions that are involved in complex actions (Graziano 2016) and which project to internal organs (Dum et al., 2018). This connectivity suggests that anterior motor regions in macaque may be analogous to the inter-effector regions in humans.

FIG. 34B depicts a schematic illustrating proposed analogous regions in macaque (left) and in human (right). The foot region (green) is displaced in the human to the medial wall, while hand (cyan) and face (orange) regions maintained their relative positions. Anterior precentral regions in macaque (maroon) were displaced posteriorly in humans into the posterior bank of the precentral gyrus, interleaved between the foot, hand, and face regions.

### Similarities Between Human Inter-Effector Motif and Non-Human Primate Anterior M1

When the PFM functional connectivity data was examined from a macaque (FIG. 34A), seeds in the posterior bank of precentral gyrus M1 revealed foot, hand, and mouth effector-specific functional connectivity patterns consistent with those seen in humans. Anterior precentral gyrus regions revealed functional connectivity with each other and rostral cingulate zone, pre-SMA, and parietal cortex that appeared analogous to the human CON (FIG. 34A).

In macaques, distinct patterns of corticospinal connectivity distinguish anterior from posterior M1. Phylogenetically newer, posterior M1 represents the effectors projects contralaterally, mainly to the cervical and lumbar enlargements of the spinal cord, and contains more projections that synapse directly onto muscle-innervating spinal neurons for fine motor control. In contrast, older anterior M1 represents both the body and more complex actions, projects bilaterally throughout the spinal cord, and connects to internal organs such as the adrenal medulla and stomach. Non-human primate electrophysiological studies reporting action planning signals often record from sites overlapping with the proposed anteriorly located macaque inter-effector and CON analogues. Thus, direct stimulation, electrophysiological recording, structural and functional connectivity data all reveal similarities between macaque anterior motor cortex and the human inter-effector motif (FIG. 34B).

### The Interrupted Homunculus—Integrate/Isolate Model of Behavioral and Motor Control

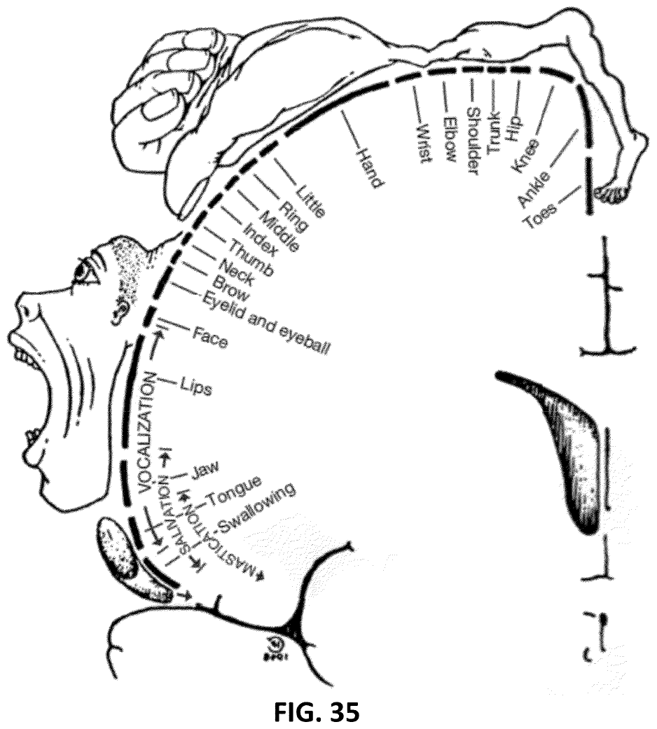

FIG. 35 depicts Penfield's classical homunculus depicting a continuous map of the body in primary motor cortex.

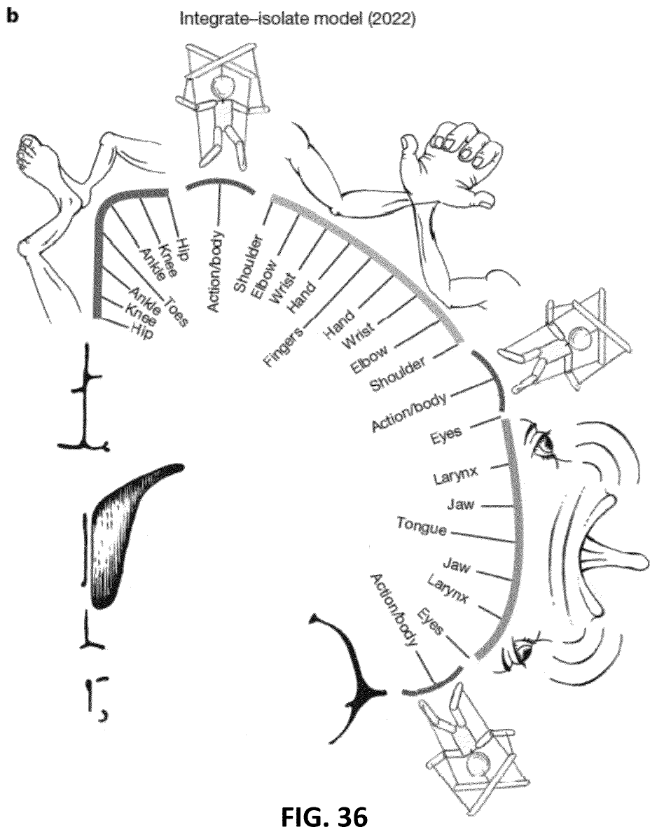

As depicted in FIG. 36, in the integrate-isolate model of primary motor cortex organization, effector-specific (foot [green], hand [cyan], mouth [orange]) functional zones are represented by concentric rings with proximal body parts surrounding the relatively more isolatable distal ones (toes, fingers, tongue). Inter-effector regions (maroon) sit at the intersecting points of these fields, forming part of a Mind-Body Interface (MBI) for integrative, allostatic whole-body control.

### Distinct Systems for Effector Isolation and Integrated Action Interleave in Motor Cortex

Penfield conceptualized his direct stimulation findings in M1 as a continuous map of the human body—the homunculus—an organizational principle that has been dominant for almost 100 years (FIG. 35). Based on novel and extant data, a dual-systems, integrate-isolate model of behavioral control, is proposed in which effector isolating and whole-organism action implementation regions alternate (FIG. 36). This model better fits the human imaging data presented here demonstrating contrasting structural, functional and connectivity patterns within M1. The inter-effector patterning emerges in infancy and is preserved even in the presence of substantial perinatal cortical injury (FIG. 12). In the integrate-isolate model, the regions for foot/hand/mouth fine motor skill are organized somatotopically as three concentric functional zones with distal parts of the effector (toes, fingers, tongue) at the center and proximal ones (knee, shoulder, jaw) on the perimeter. The inter-effector regions at the edges of the effector zones coordinate with each other and the CON to accomplish holistic, whole-body functions in the service of performing actions. The present work suggests these functions include action implementation, as well as postural and gross motor control of axial muscles, while prior work in humans and non-human primates suggests these circuits may also regulate arousal and control of internal processes and organs (i.e., blood pressure, stomach, adrenal medulla), consistent with circuits for whole-body, metabolic, and physiological control. Thus, the inter-effector system fulfills the role of a Mind-Body Interface (MBI). The MBI forms part of a unified action control system, in conjunction with the CON's upstream executive control operations, to coordinate gross movements and muscle groups (e.g., torso, eyebrow), and enact top-down control of posture and internal physiology, while preparing for actions. These proposed functions converge with the concept of allostatic regulation, by which the brain anticipates upcoming changes in physiological demands based on planned actions and exerts top-down preparatory control over the body.

### Human Stimulation Evidence for the Integrate-Isolate Patterning of Motor Cortex

Penfield proposed the homunculus as an approximation of group-averaged, intraoperative direct electrocortical stimulation data, which showed significant overlap across patients and body parts. He later described his artistic rendering of the homunculus as “an aid to memory [ . . . ] a cartoon of representation in which scientific accuracy is impossible”. Re-examination of extant human stimulation data raises doubts about the veracity of the homunculus in individuals and reveals an equal or better fit with the integrate-isolate model. In some patients, a distal-to-proximal concentric organization was documented for the upper limb, just as in non-human primates, while face movements could be elicited from areas dorsal to the hand representation. In addition to focal movements, several other response types are routinely elicited with M1 stimulation, all of which can be better accounted for by whole-organism control regions. Patients have reported the urge to move, while being aware that they are holding still; they have reported a sense of moving though no movement is detectable; or they have moved but denied having done so-effects consistent with modulation of a system also representing action goals. These responses are similar to those typically elicited in CON regions such as dACC and anterior parietal cortex.

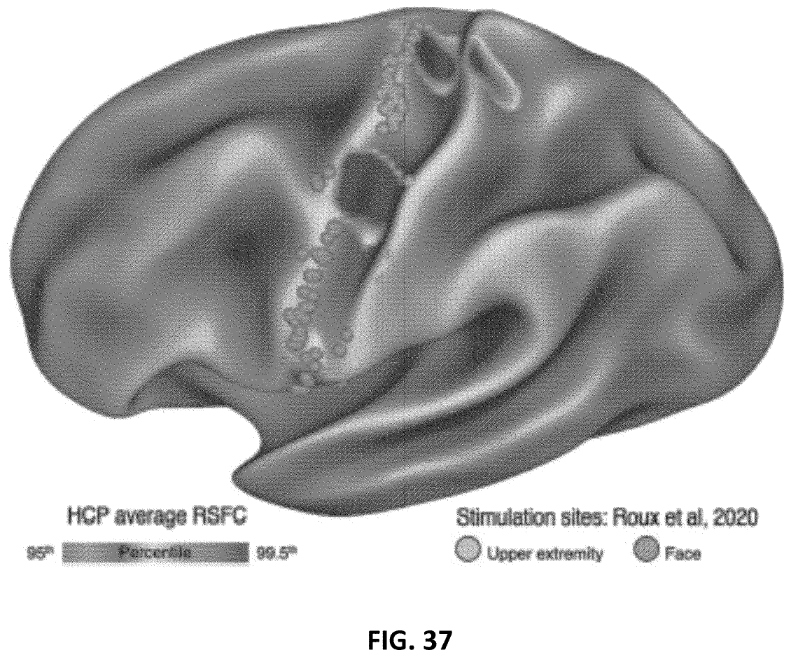

Stimulations almost never produce isolated torso or shoulder movements, and a common outcome of stimulation is no reported response at all. Historically, stimulations failing to elicit movement were not documented. However, the motor stimulations were re-analyzed from a recent large study by mapping them onto cortex, revealing a region that never elicited movement in any patient, corresponding to the middle inter-effector region (FIG. 37). These results suggest that stimulation strengths deemed safe in humans may not typically elicit movements in the M1 Mind-Body Interface regions, akin to higher-order lateral and medial premotor (i.e., pre-SMA) regions. Human brain-computer-interface (BCI) recordings in M1 have also demonstrated whole-body movement tuning, possibly reflecting inter-effector activity and suggesting that the inter-effector motif could provide a target for whole-body BCI.

### Inter-Effector Regions in Human Cortical Stimulation and Prior Network Mapping Work

The map of inter-effector regions was compared with published movements evoked by direct cortical stimulation. Cortical map: functional connectivity is shown seeded from the central inter-effector M1 region and averaged across all subjects in the HCP dataset (see also FIG. 8). Stimulation locations: MNI coordinates of stimulation location, and the resulting evoked movement, from 100 patients undergoing awake surgical brain mapping were reported in. Each stimulation location evoking any movement was mapped to the nearest cortical vertex on a group average pial surface. Stimulation sites are colored according to whether they evoked facial movements (orange) or upper extremity movements (cyan). Stimulation sites evoking movement did not overlap with the central inter-effector region.

### Clinical Evidence for Separate Movement Isolation and Integrated Action Control Systems

Brain lesion data further support the existence of dual systems for movement isolation and action integration, with partial redundancy in M1. Motor deficits after middle cerebral artery strokes are unilateral, more severe in most distal effectors, and without significant global organismal control deficits. By contrast, lesions of MBI-linked CON regions (dACC, anterior insula, aPFC) can cause isolated volitional deficits ranging from decreased fluency to abulia to akinetic mutism, with preserved motor abilities but little self-generated movement. Similarly, anterior motor lesions in macaques can spare visually guided movements while selectively disrupting internally generated actions. Animals with lesions in effector M1 typically recover gross effector control very quickly, while fine finger movement deficits persist longer. More rapid recovery of gross motor abilities may be in part due to proximal functions being taken up by the contra-lesional MBI circuits, enabled by their bilateral spinal cord connectivity. Persistent deficits may therefore be more likely in functions uniquely supported by the effector-specific circuitry.

In a proof-of-principle patient with extensive bilateral perinatal strokes but typical motor ability, extensive post-stroke reorganization maintained the MBI patterning at the cost of sacrificing part of the already reduced M1 hand area. The top third of M1 was destroyed, and surviving cortex contained an M1 hand area that was ventrally shifted and much smaller than in typical controls. Surprisingly, MBI regions were identified both above and below the surviving effector-specific hand region (FIG. 12F), highlighting the Mind-Body Interface's importance for typical motor ability.

With specific connections to thalamic motor nuclei used as targets for clinical intervention (VIM, CM), the CON-linked MBI may be relevant for a variety of movement disorders, including dystonia or essential tremor. Parkinson's Disease (PD) is of particular note. Many PD symptoms cut across motor, physiological and volitional domains (e.g., postural instability, autonomic dysfunction, and reduced self-initiated activity, among many others), mirroring MBI connections to regions relevant for postural control (cerebellar vermis), volition, and physiological regulation (CON).

### Sensory and Motor Systems Share Common Functional Organization Features

Many of the motor cortex organizational features described here have clear parallels in sensory systems. Like the concentric somatotopic organization with fine finger movements at the center, primary visual cortex over-represents higher acuity processing at the center, concentrically transitioning to lower acuity in the periphery. Like our integrate/isolate dual-systems model, visual processing streams are parallel and separated in thalamus, early visual cortex, and higher order visual processing streams, with each level of processing maintaining segregation of different types of information (e.g., early: eccentricity vs. angle; late: faces vs. objects). Auditory processing may have similar features, as acoustic signals are processed at least partially in parallel for hearing and speech perception in superior temporal gyrus. These findings suggest shared organizational principles across the brain's input and output processing streams. It is possible S1 may also have some concentric organizational elements, which should be explored in future work.

### A Network for Mind-Body Integration

Two behavioral control systems are interleaved in human motor cortex. One well-known system consists of effector-specific circuits for precise, isolated movements of highly specialized appendages—fingers, toes, and tongue—the type of dexterous motion needed for speaking or manipulating objects. A second, integrative output system, the Mind-Body Interface (MBI) is more important for controlling the organism as a whole. The MBI integrates body control (motor and autonomic) and action planning, consistent with the idea that aspects of higher-level executive control might derive from movement coordination. The MBI includes specific regions of M1, the SMA, thalamus (VIM, CM), posterior putamen, and the postural cerebellum and is functionally connected to the dACC, which has been linked to free will, parietal regions representing movement intentions, insular regions for processing somatosensory, pain, and interoceptive visceral signals. A common factor across this fairly wide range of processes is that they must be integrated if an organism is to achieve its goals through movement while avoiding injury and maintaining physiological allostasis. The MBI provides a substrate for this integration, enabling pre-action anticipatory postural, cardiovascular, and arousal changes (e.g., shoulder tension, increased heart rate, butterflies in the stomach). The discovery that action and body control are melded in a common circuit could help explain why mind and body states so often interact.

### Declaration of Interests:

DAF and NUFD have a financial interest in NOUS Imaging Inc. and may financially benefit if the company is successful in marketing FIRMM motion-monitoring software products. DAF and NUFD may receive royalty income based on FIRMM technology developed at Washington University School of Medicine and Oregon Health and Sciences University and licensed to NOUS Imaging Inc. DAF and NUFD are co-founders of NOUS Imaging Inc. These potential conflicts of interest have been reviewed and are managed by Washington University School of Medicine, Oregon Health and Sciences University and the University of Minnesota. The other authors declare no competing interests.

### Methods

### Subjects

### Individual-Specific PFM Data—Washington University Adults

### Participants:

Data were collected from three healthy, right-handed, adult participants (ages 35, 25, and 27; 1 female) as part of a study investigating effects of arm immobilization on brain plasticity (data previously published in). Two of the participants are authors (NUFD and ANN). Informed consent was obtained from all participants. The study was approved by the Washington University School of Medicine Human Studies Committee and Institutional Review Board. The primary data employed here was collected either prior to the immobilization intervention (Participants 01, 03) or two years afterwards (Participant 02). Data collected immediately after the intervention is presented for within-participant replication in FIG. 11.

In two participants (Participants 01, 02), additional fMRI data was collected using the same sequence during performance of two motor tasks: a somatotopic mapping task and a motor control task.

### Somatotopic Mapping Task

A block design was adapted. In each run, the participant was presented with visual cues that directed them to perform one of five specific motor movements. Each block started with a 2.2 s cue indicating which movement was to be made. After this cue, a centrally-presented caret replaced the instruction and flickered once every 1.1 s (without temporal jittering). Each time when the caret flickered, participants executed the proper movement. 12 movements were made per block. Each block lasted 15.4 seconds, and each task run consisted of 2 blocks of each type of movement as well as 3 blocks of resting fixation. Movements conducted within each run were as follows:

| | | | | | # of runs |
|---|---|---|---|---|---|
| Run Type 1 | Run Type 2 | Run Type 3 | Run Type 4 | Run Type 5 | (P1/P2) |
| Close L hand | Close R hand | Flex L Foot | Flex R Foot | Wiggle | 24 / 20 |
| | | | | Tongue | |
| Flex L elbow | Flex R Elbow | Flex L Wrist | Flex R Wrist | Lift bilat | 10 / 11 |
| | | | | Shoulders | |
| Flex L | Flex R Gluteus | Tense | Open/Close | Swallow | 10 / 11 |
| Gluteus | | Abdomen | Mouth | | |
| Flex L Ankle | Flex R Ankle | Bend L Knee | Bend R Knee | Flex bilat | 10 / 11 |
| | | | | Toes | |
| Lift L | Lift R Eyebrow | Wink L Eyelid | Wink R Eyelid | Flare Nostrils | 10 / 11 |
| Eyebrow | | | | | |

### Action Control and Coordination Task

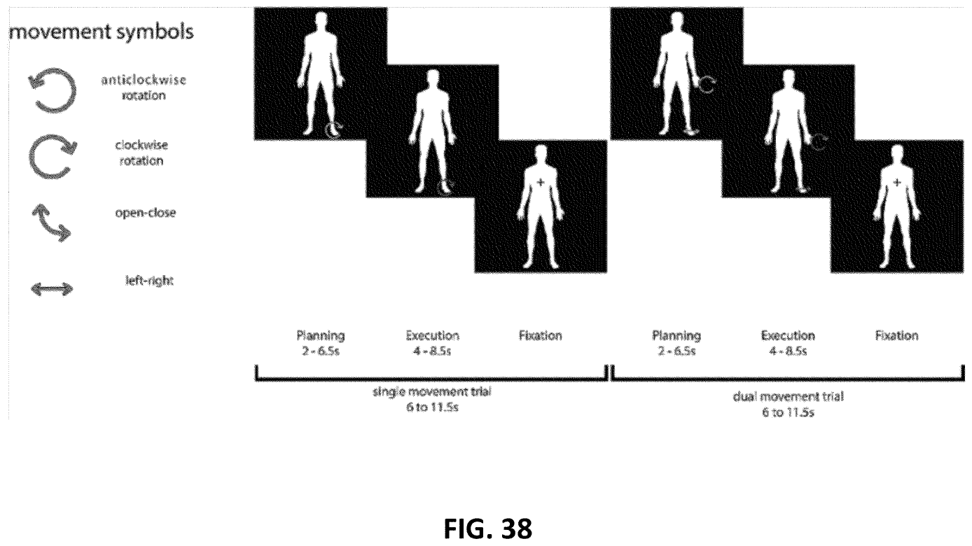

An event-related design implemented using JSpsych toolbox v6.3 was used to discriminate planning and execution of limb movement. Within the run, the participant is prompted to move either a single limb or to simultaneously move two limbs. Referring to FIG. 38, there are four possible motions—open-close of fingers or toes, left-right flexion of the wrist/ankle, clockwise rotation of the wrist/ankle, and counterclockwise rotation of the wrist/ankle—each of which may be executed by any of the four extremities (left or right upper/lower extremity). Each motion/extremity combination may be required in isolation, or in combination with a second simultaneous motion. The participant is cued to prepare the movement(s) when they see one or two movement symbols placed on a body shape in a grey color (planning phase), and is then cued to execute the movement(s) when the grey symbol or symbols turn green (execution phase). Using a random jitter, the planning phase can last from 2 to 6.5 s followed by 4 to 8.5 s of movement execution. Each movement trial (planning and execution) is followed by a jittered fixation of up to 5 s. A rest block of 8.6 s is implemented every 12 movements. Two possible movements are requested during the task run and practiced before the task. The movement pair is changed for each task run. Twelve total runs were acquired per participant.

### Individual-Specific PFM Data—Cornell Adults

### Participants:

Data were collected from four healthy adult participants (ages 29, 38, 24, and 31; all male) as part of a previously published study. Two of the participants are authors (CJL and JDP). The study was approved by the Weill Cornell Medicine Institutional Review Board.

### Individual-Specific PFM Data—Neonate

### Participant:

Data were collected from one sleeping, healthy full-term neonatal participant beginning 13 days after birth, corresponding to 42 weeks post-menstrual age. The study was approved by the Washington University School of Medicine Human Studies Committee and Institutional Review Board.

### MRI Acquisition:

The participant was scanned while asleep over the course of 4 consecutive days using a Siemens Prisma 3T scanner on the Washington University Medical Campus. Every session included collection of a high-resolution T2-weighted spin-echo image (TE=563 ms, TR=3200 ms, flip angle=120°, 208 slices with 0.8×0.8×0.8 mm voxels). In each session, a number of 6 minute 45 second multi-echo resting-state fMRI runs were collected as a five-echo blood oxygen level-dependent (BOLD) contrast sensitive gradient echo-planar sequence (flip angle=68°, resolution=2.0 mm isotropic, TR=1761 ms, multiband 6 acceleration, TE₁: 14.20 ms, TE₂: 38.93 ms, TE₃: 63.66 ms, TE₄: 88.39 ms, and TE₅: 113.12 ms). The number of BOLD runs collected in each session depended on the ability of the neonate to stay asleep during that scan; across the four days, 23 runs were collected in total. A pair of spin echo EPI images with opposite phase encoding directions (AP and PA) but identical geometrical parameters and echo spacing were acquired between every 3 BOLD runs or any time the participant was removed from the scanner.

### MRI Processing

Structural and functional processing followed the pipeline used for the Wash U dataset, with two exceptions. First, segmentation, surface delineation, and atlas registration were conducted using a T2-weighted image (the single highest quality T2 image, as assessed via visual inspection) rather than a T1-weighted image, due to the inverted image contrast observed in neonates. Second, after the multi-echo BOLD data was unwarped and normalized to atlas space, it was optimally combined before nuisance regression and mapping to cifti space. All fMRI scans from the second day of scanning were excluded due to registration abnormalities.

### Individual-Specific PFM Data—Infant

### Participant:

Data were collected from one healthy sleeping infant at the age of 11 months. The study was approved by the Washington University School of Medicine Human Studies Committee and Institutional Review Board.

### MRI Acquisition:

The participant was scanned while asleep over the course of three sessions using a Siemens Prisma 3T scanner on the Washington University Medical Campus. The first session included collection of a high-resolution T1-weighted MP-RAGE (TE=2.24 ms, TR=2400 ms, flip angle=8°, 208 slices with 0.8×0.8×0.8 mm voxels) and a T2-weighted spin-echo image (TE=564 ms, TR=3200 ms, flip angle=120°, 208 slices with 0.8×0.8×0.8 mm voxels). The second and third sessions included collection of 26 total runs of resting-state fMRI, each collected as a 6 minute 49 second long blood oxygen level-dependent (BOLD) contrast sensitive gradient echo-planar sequence (flip angle=52°, resolution=3.0 mm isotropic, TE=30 ms, TR=861 ms, multiband 4 acceleration). For each run, a pair of spin echo EPI images with opposite phase encoding directions (AP and PA) but identical geometrical parameters and echo spacing were acquired to correct spatial distortions.

### MRI Processing

Structural processing followed the DCAN Labs processing pipeline (https://github.com/DCAN-Labs/abed-hep-pipeline), which was found to perform the best surface segmentation at this age. Functional processing followed the pipeline used for the Wash U dataset.

### Individual-Specific PFM Data—Child

### Participant:

Data were collected from one healthy awake male child age 9 yo. The study was approved by the Washington University School of Medicine Human Studies Committee and Institutional Review Board.

### MRI Acquisition:

The participant was scanned repeatedly over the course of 12 sessions using a Siemens Prisma 3T scanner on the Washington University Medical Campus. These sessions included collection of 14 high-resolution T1-weighted MP-RAGE images (TE=2.90 ms, TR=2500 ms, flip angle=8°, 176 slices with 1 mm isotropic voxels), 14 T2-weighted spin-echo images (TE=564 ms, TR=3200 ms, flip angle=120°, 176 slices with 1 mm isotropic voxels), and 26 total runs of resting-state fMRI, each collected as a 10 minute long blood oxygen level-dependent (BOLD) contrast sensitive gradient echo-planar sequence (flip angle=84°, resolution=2.6 mm isotropic, 56 slices, TE=33 ms, TR=1100 ms, multiband 4 acceleration). In each session, a pair of spin echo EPI images with opposite phase encoding directions (AP and PA) but identical geometrical parameters and echo spacing were acquired to correct spatial distortions in the BOLD data.

### MRI Processing

Structural and functional processing followed the DCAN Labs processing pipeline (https://github.com/DCAN-Labs/abcd-hcp-pipeline).

### Individual-Specific PFM Data—Perinatal Stroke

### Participant:

PS1, a left-handed, 13-year-old male who played for a competitive youth baseball team, was referred to an orthopedic physician because of difficulty using his right arm effectively. Ulnar neuropathy was considered and he was referred for physical therapy. However, PS1 was first seen by a child neurologist (NUFD) for further evaluation. Structural brain MRI revealed unexpectedly extensive bilateral cystic lesions consistent with perinatal infarcts. Review of PS1's medical history revealed that the injury occurred in the perinatal period.

Data acquisition from PS1 were performed with the approval of the Washington University Institutional Review Board. Written informed consent was provided by PS1's mother and assent was given by PS1 at the time of data acquisition.

For additional details regarding clinical history, neuropsychological evaluations, motor assessments, or MR image acquisition or processing, see ³⁵.

### Individual-Specific PFM Data—Macaque

### Animal Preparation:

Data were collected from a sedated adult female macaque monkey (Macaca fascicularis). Experimental procedures were carried out in accordance with the University of Minnesota Institutional Animal Care and Use Committee and the National Institute of Health standards for the care and use of non-human primates. The subject was fed ad libitum and pair-housed within a light and temperature-controlled colony room. The animal was not water restricted. The subject did not have any prior implant or cranial surgery. The animal was fasted for 14-16 hr prior to imaging. On scanning days, anesthesia was first induced by intramuscular injection of atropine (0.5 mg/kg), ketamine hydrochloride (7.5 mg/kg), and dexmedetomidine (13 g/kg). The subject was transported to the scanner anteroom and intubated using an endotracheal tube. Initial anesthesia was maintained using 1.0%-2% isoflurane mixed with oxygen (1 L/m during intubation and 2 L/m during scanning to compensate for the 12-m length of the tubing used). For functional imaging, the isoflurane level was lowered to 1%. The subject was placed onto a custom-built coil bed with integrated head fixation by placing stereo-tactic ear bars into the ear canals. The position of the animal corresponds to the sphinx position. Experiments were performed with the animal freely breathing. Continuous administration of 4.5 g/kg/hr dexmedetomidine using a syringe pump was administered during the procedure. Rectal temperature (˜99.6 F), respiration (10-15 breaths/min), end-tidal CO 2 (25-40), electro-cardiogram (70-150 bpm), and SpO2 (>90%) were monitored using an MRI compatible monitor (IRAD-IMED 3880 MRI Monitor, USA). Temperature was maintained using a circulating water bath as well as chemical heating pads and padding for thermal insulation.

### MRI Acquisition:

Data were acquired on a Simens Magnetom 10.5 T Plus. A custom in-house built and designed RF coil was used with an 8-channel transmit/receive end-loaded dipole array of 18-cm length combined with a close-fitting 16-channel loop receive array head cap, and an 8-channel loop receive array of 50×100 mm² size located under the chin. A B1+ (transmit B1) fieldmap was acquired using a vendor provided flip angle mapping sequence and then power calibrated for each individual. Following B1+transmit calibration, 3-5 averages (23 min) of a T1 weighted MP-RAGE were acquired for anatomical processing (TR=3300 ms, TE=3.56 ms, TI=1140, flip angle=5°, slices=256, matrix=320×260, acquisition voxel size=0.5×0.5×0.5 mm 3, in-plane acceleration GRAPPA=2). A resolution and FOV matched T2 weighted 3D turbo spin-echo sequence was run to facilitate B1 inhomogeneity correction. Five images were acquired in both phase-encoding directions (R/L and L/R) for offline EPI distortion correction. Six runs of fMRI timeseries, each including 700 continuous 2D multiband EPI functional volumes (TR=1110 ms; TE=17.6 ms; flip angle=60°, slices=58, matrix=108×154; FOV=81×115.5 mm; acquisition voxel size=0.75×0.75×0.75 mm) were acquired with a left-right phase encoding direction using in plane acceleration factor GRAPPA=3, partial Fourier=7/8th, and MB factor=2. Since the macaque was scanned in sphinx position, the orientations noted here are what is consistent with a (head first supine) typical human brain study (in terms of gradients) but translate differently to the actual macaque orientation.

### MRI Processing

Processing followed the DCAN Labs non-human primate processing pipeline (https://github.com/DCAN-Labs/nhp-abcd-bids-pipeline), with minor modifications. Specifically, it was observed that distortion from the 10T scanner was so extensive that the field maps did not fully correct it. Therefore, instead of field-map based unwarping, the computed field map-based warp was used as an initial starting point for Synth, a field map-less distortion correction algorithm that creates synthetic undistorted BOLD images for registration to anatomical images. Synth substantially reduced residual BOLD image distortion.

### Group-Averaged Datasets

Resting-state fMRI data was averaged across participants within each of five large datasets.

### UKB:

A group-average weighted eigenvectors file from an initial batch of 4100 UKB participants scanned using resting-state fMRI for six minutes was downloaded from https://www.fmrib.ox.ac.uk/ukbiobank/. This file consisted of the top 1200 weighted spatial eigenvectors from a group-averaged PCA. See and documentation at https://biobank.etsu.ox.ac.uk/crystal/ukb/docs/brain_mri.pdf for details of the acquisition and processing pipeline. This eigenvectors file was mapped to the Conte69 surface template atlas using the ribbon-constrained method in Connectome Workbench, and the eigenvector timecourses of all surface vertices were cross-correlated.

### ABCD:

Twenty minutes (4×5 minute runs) of resting-state fMRI data, as well as high-resolution T1-weighted and T2-weighted images, were collected from 3,928 9-10 year old participants, who were selected as the participants with at least 8 minutes of low-motion data from a larger scanning sample. Data collection was performed across 21 sites within the Unites States, harmonized across Siemens, Philips, and GE 3T MRI scanners. Data processing was conducted using the ABCD-BIDS pipeline (https://github.com/DCAN Labs/abcd-hcp-pipelines).

### HCP:

A vertexwise group-averaged functional connectivity matrix from the HCP 1200 participants release was downloaded from db.humanconnectome.org. This matrix consisted of the average strength of functional connectivity across all 812 participants who completed four 15-minute resting-state fMRI runs and who had their raw data reconstructed using the newer “recon 2” software.

### WashU:

Data was collected from 120 healthy young adult participants recruited from the Washington University community during relaxed eyes-open fixation (60 females, mean age=25 years, age range=19-32 years). Scanning was conducted using a Siemens TRIO 3.0T scanner and included collection of high-resolution T1-weighted and T2-weighted images, as well as an average of 14 minutes of resting-state fMRI. See for details of the acquisition and processing pipeline.

Neonates (eLABE):

Mothers were recruited during the 2nd or 3rd trimester from two obstetrics clinics at Washington University as part of the Early Life Adversity, Biological Embedding, and Risk for Developmental Precursors of Mental Disorders (eLABE) study. Neuroimaging was conducted in full-term, healthy neonate offspring shortly after birth (average post-menstrual age of included participants 41.4 weeks, range 38-45). Of the 385 participants scanned for eLABE, 262 were included in the current analyses. See for additional details of the participants, criteria for exclusion, scanning acquisition protocol and parameters, and processing pipeline.

### Analyses

### Functional Connectivity

For each single-participant dataset, a vertex/voxelwise functional connectivity matrix was calculated from the resting-state fMRI data as the Fisher-transformed pairwise correlation of the timeseries of all vertices/voxels in the brain. In the ABCD, WashU120, and eLABE datasets, vertex/voxelwise group-averaged functional connectivity matrices were constructed by first calculating the vertex/voxelwise functional connectivity within each participant as the Fisher-transformed pairwise correlation of the timeseries of all vertices/voxels in the brain, and then averaging these values across participants at each vertex/voxel.

### Seed-Based Functional Connectivity

A continuous line of seeds was defined down the left precentral gyrus by selecting every vertex in a continuous straight line on the cortical surface between the most ventral aspect of the medial motor area (approximate MNI coordinates [−4, −31, 54]) and the ventral lip of the precentral gyrus right above the operculum (approximate MNI coordinates [−58 4 8]). For each seed, its map of functional connectivity was examined as the Fisher-transformed correlation between that vertex's timecourse and that of every other vertex/voxel in the brain.

### Network Detection in Somatomotor Cortex

To define the somatomotor regions that were visually identified from the seed-based connectivity analysis in an unbiased fashion for further exploration, each individual adult human participant's data was entered into a data-driven network detection algorithm designed to identify network subdivisions that are hierarchically below the level of classic large-scale networks (e.g. that produce hand/foot divisions in somatomotor cortex). This approach identifies sub-network structures that converge with task-activated regions and with known neuroanatomical systems.

In each adult participant, this analysis clearly identified network structures corresponding to motor representation of the foot, hand, and mouth; and it additionally identified network structures corresponding exactly to the previously unknown connectivity pattern identified from the seed-based connectivity exploration as the inter-effector regions. For simplicity, all inter-effector subnetworks were manually grouped together as a single putative network structure (labeled as inter-effector) for further analysis.

Finally, to identify classic large-scale networks in each participant, the Infomap algorithm was reran on matrices thresholded at a series of denser thresholds (ranging from 0.2% to 5%), and additionally identified individual-specific networks corresponding to the Default, Medial and Lateral Visual, Cingulo-opercular, Fronto-Parietal, Dorsal Attention, Language, Salience, Parietal Memory, and Contextual Association networks.

### Differences in Functional Connectivity Between Inter-Effector and Foot/Hand/Mouth Regions

Within each adult human participant, an inter-effector connectivity map was calculated as the Fisher-transformed correlation between the average timecourse of all cortical inter-effector vertices and the timecourse of every other vertex/voxel in the brain. The procedure is repeated to calculate a connectivity map for the foot, hand, and mouth areas.

To identify brain regions more strongly connected to inter-effector regions than to other motor regions, the smallest positive difference is computed in each voxel/vertex between inter-effector connectivity and any foot/hand/mouth connectivity. This represents a conservative approach that only identifies regions of the brain for which the inter-effector regions are more strongly connected than any of the other motor areas.

Functional Connectivity with CON

Within each adult human participant, the functional connectivity was calculated between each of the foot, hand, mouth, and inter-effector regions and the CON. This was computed as the Fisher-transformed correlation between 1) the average timecourse across all vertices in the motor region and 2) the average timecourse across all vertices in the CON. Paired t-tests are conducted across subjects comparing the inter-effector connectivity with CON against each of the foot/hand/mouth connectivities, correcting for the three tests conducted.

### Motor/CON Network Visualization

Visualization of network relationships was conducted using spring-embedded plots, as implemented in Gephi (https://gephi.org/). In each individual adult human participant, nodes were defined as congruent clusters of foot, hand, mouth, inter-effector, and CON networks larger than 20 mm². Pairwise connectivity between nodes was calculated as the Fisher-transformed correlation of their mean timecourses. For visualization purposes, graphs were constructed by thresholding the pairwise node-to-node connectivity matrices at 40% density (the general appearance of the graphs did not change across a range of densities).

Functional Connectivity with Adjacent Postcentral Gyrus

In each adult human participant, the pre- and postcentral gyri are defined based on the individual-specific Brodmann areal parcellation produced by Freesurfer, which was deformed into fs_LR_32k space to match the functional data. Precentral gyrus was considered to be the vertices labeled as Brodmann Areas 4a and 4p, while postcentral gyrus was the vertices labeled as Brodmann Areas 3b and 2. Brodmann area 3a (fundus of central sulcus) was not considered for this analysis. Because the medial aspect of somatomotor cortex (corresponding to representation of the leg and foot) was always classified by Freesurfer as BA 4a, the medial postcentral gyrus are defined as the cortical vertices with y-coordinates farther posterior than the median y-coordinate of the foot region (from the network mapping above).

Within the participant's precentral gyrus, vertices are labeled as representing foot, hand, mouth, or inter-effector according to their labels from the network mapping procedure. The postcentral gyrus were partitioned into foot, hand, mouth, and inter-effector areas depending on which precentral region each vertex was physically closest to. Finally, within each partition (foot, hand, mouth, and inter-effector) the average connectivity is calculated between the pre and postcentral gyrus as the Fisher-transformed correlation between the average timecourses of all vertices in each area. Paired t-tests were conducted across subjects comparing the inter-effector connectivity with adjacent S1 against each of the foot/hand/mouth connectivities with S1, FDR-correcting for the three tests conducted.

Functional Connectivity with Middle Insula

In each adult human participant, the middle insula was defined based on the individual-specific Freesurfer gyral parcellation using on the Destrieux atlas, which was deformed into fs_LR_32k space to match the functional data. Middle insula was considered to be the vertices labeled as the superior segment of the circular sulcus of the insula or as the short insular gyrus. The functional connectivity was calculated between each of the bilateral foot, hand, mouth, and inter-effector regions and the bilateral middle insula. Paired t-tests were conducted across subjects comparing the inter-effector connectivity with middle insula against each of the foot/hand/mouth connectivities, FDR-correcting for the number of tests conducted.

Functional Connectivity with Cerebellum

In each adult human participant, the functional connectivity was calculated between each of the foot, hand, mouth, and inter-effector regions with each voxel of the cerebellum. Cerebellar connectivity strengths calculated this way were then mapped onto a cerebellar flat map using the SUIT toolbox. Connectivity strengths were averaged within each of 27 atlas regions. For each region, three paired t-tests were conducted comparing inter-effector connectivity strength against foot, hand, and mouth connectivity strength, FDR-correcting for the total number of tests conducted. Regions were reported if the inter-effector connectivity strength was significantly higher than the connectivity strength of all other motor regions.

Functional Connectivity with Putamen

In each adult human participant, each unilateral putamen was divided in each hemisphere into quarters by splitting it based on the median of its Y (anterior-posterior) and Z (dorsal-ventral) coordinates. The functional connectivity was calculated between each of the foot, hand, mouth, and inter-effector regions and each putamen quarter.

For each putamen division, paired t-tests were conducted across subjects comparing the inter-effector connectivity with that putamen division against each of the foot/hand/mouth connectivities, FDR-correcting for the number of tests conducted. Divisions were reported in which the inter-effector connectivity was significantly different from all three effector-specific connectivities.

Functional Connectivity with Thalamus

To investigate subregions of thalamus, the DISTAL atlas v1.1, was employed which contains a number of histological thalamic subregions identified by. This atlas was downsampled into the 2 mm isotropic space of the functional data. Functional connectivity maps seeded from the foot, hand, mouth, and inter-effector regions in each adult human participant were computed, and mean connectivity values were calculated within each atlas region. The atlas specifies multiple subregions for many nuclei; these subregions were combined and treated as single nuclei for the purposes of connectivity calculation.

For each adult human participant, the connectivity seeded was averaged from the inter-effector regions and from each of the foot, hand, and mouth regions across all voxels within each thalamic nucleus. For each thalamic nucleus, paired t-tests were conducted across subjects comparing the inter-effector with the mean of the foot/hand/mouth connectivities, bonferroni correcting for the number of thalamic nuclei tested.

### Lag Structure of RSFC

A previously published method was used for estimating relative time delays (“lags”) in fMRI data. Briefly, for each session in each adult human participant, a lagged cross-covariance function (CCF) was computed between each pair of vertex/voxel timecourses within the motor system and CON in the cortex. Lags were more precisely determined by estimating the cross-covariance extremum of the session-level CCF using three-point parabolic interpolation. The resulting set of lags was assembled into an antisymmetric matrix capturing all possible pairwise time delays (TD matrix) for each session, which was averaged across sessions to yield participant-level TD matrices. Finally, each participant's TD matrix was averaged across rows to summarize the average time-shift from one vertex to all other vertices. Average time lag was then averaged across all vertices with each of the precentral gyrus foot/hand/mouth/inter-effector regions, and the CON.

Paired t-tests were conducted across subjects comparing 1) the mean lag in inter-effector regions against the mean lags in each of the foot/hand/mouth regions, and 3) the mean lag in CON regions against the mean lags in each of the foot/hand/mouth regions.

### Structural MRI

### Cortical Thickness

Within each adult human participant, the map of cortical thickness generated by the Freesurfer segmentation was deformed into fs_LR_32k space to match the functional data. Precentral gyrus foot, hand, mouth, and inter-effector regions were defined as above, and mean cortical thickness was calculated within each region. Paired t-tests were conducted across subjects comparing the inter-effector thickness against each of the foot/hand/mouth thicknesses, correcting for the three tests conducted.

### Fractional Anisotropy

White matter fibers tracked from separate areas of motor cortex using diffusion imaging quickly converge into the internal capsule and become difficult to dissociate. As such, FA differences were tested for in the white matter immediately below the precentral gyrus.

To calculate FA beneath the cortex, fs_LR_32k-space surfaces were constructed 2 mm below each gray-white surface in adult human participants P1-P3. To accomplish this, for each vertex on the surface, the 3D vector was computed between corresponding points on the fs_LR_32k pial and the gray-white surfaces, and that vector was extended an additional 2 mm beyond the gray-white surface in order to create a lower surface. The FA values were then mapped using the using the ribbon-constrained method, mapping between the gray-white and the 2 mm-under surfaces. The result is FA values mapped to a lower surface within white matter that is in register to the existing fs_LR_32k surfaces on which the functional data is mapped and the motor regions defined.

Precentral gyrus foot, hand, mouth, and inter-effector regions were defined as above, and mean FA were calculated beneath each cortical region.

Paired t-tests were conducted across subjects comparing the mean FA beneath the inter-effector regions against mean FA beneath each of the foot/hand/mouth thicknesses.

### Myelin Density

Within each adult human participant, vertexwise maps were created of intracortical myelin content. Precentral gyrus was defined as above. Across participants, it was found that baseline myelin density values (both in precentral gyrus and in the whole-brain myelin density map) varied wildly across participants in different datasets, likely based on differences in the T1- and T2-weighted sequences employed. Thus, for optimal visualization of results, in each participant, the myelin density values were normalized by dividing the calculated vertexwise myelin densities in precentral gyrus by the mean myelin density across the whole precentral gyrus. Finally, precentral gyrus foot, hand, mouth, and inter-effector regions were defined as above, and mean normalized myelin density was calculated within each region. Paired t-tests were conducted across subjects comparing the inter-effector myelin density against each of the foot/hand/mouth myelin densities, correcting for the three tests conducted.

Task fMRI

### Somatotopic Task Activation

Basic analysis of the somatotopic task data was conducted using within-participant block designs. To compute the overall degree of activation in response to each motion, data from each run was entered into a first-level analysis within FSL's FEAT in which each motion block was modeled as an event of duration 15.4 s, and the combined block waveform for each motion condition was convolved with a hemodynamic response function to form a separate regressor in a GLM analysis testing for the effect of the multiple condition regressors on the timecourse of activity within every vertex/voxel in the brain. Beta value maps for each condition were extracted for each run and entered into a second-level analysis, in which run-level condition betas were tested against a null hypothesis of zero activation in a one-sample t-test across runs (within participant). The resulting t-values from each motion condition tested in this second-level analysis were converted to Z-scores. Z-score activation maps were smoothed with a geodesic 2D (for surface data) or Euclidean 3D (for volumetric data) Gaussian kernel of σ=2.55 mm.

### Somatotopic Winner Take all

For each vertex within the broad central sulcus area, the movement that produced the greatest activation strength (Z-score from second-level analysis, above) was identified in that vertex, and that motion was assigned to that vertex.

### Somatotopic Curve Fitting

For each vertex within precentral gyrus, its position was first computed along the dorsal-ventral axis of left hemisphere M1. This was done by identifying the closest point within the continuous line of points running down precentral gyrus (defined in Seed-based functional connectivity), and assigning that closest point's ordered position within the line to the vertex.

For every movement, that dorsal-ventral M1 position was plotted against Z-score activation in each vertex. Two curves were fit to each of these relationships. The first curve was a single-gaussian model of the form:

$$Activation = a 1 ⋆ exp ( - ( ( Position - b 1 ) / c 1 ) ⋀ 2 ) .$$

The second curve was a double-gaussian model of the form:

$$Activation = a 1 ⋆ exp ( - ( ( Position - b 1 ) / c 1 ) ⋀ 2 ) + a 2 ⋆ exp ( - ( ( Position - b 2 ) / c 2 ) ⋀ 2 ) .$$

The “a1” and “a2” parameters in each model were constrained to be positive (to enforce positive-going peaks). Curve fitting was constrained to be conducted within the general vicinity of the activated area in order to avoid fitting negative activations observed in distant portions of M1. For lower extremity movements, this meant excluding the bottom third of M1; for upper extremity movements, the bottom third of M1 plus the medial wall; for face movements, the top third of M1.

Finally, the one- or two-peak models were tested to determine which better fit the data. This was done by conducting an F-test between the models, computed as:

$$F = ( ( S S {E}_{1 peak} - S S {E}_{2 peaks} ) / ( {df}_{1 peak} - d {f}_{2 peaks} ) ) / ( SS {E}_{2 peaks} / {df}_{2 peaks} ) .$$

The p value was computed from this F by employing the F-statistic continuous distribution function (fcdf.m) in Matlab and using (df₁ₚₑₐₖ−df₂ₚₑₐₖₛ) and df₂ₚₑₐₖₛ as the numerator and denominator degrees of freedom, respectively.

### Somatotopic Selectivity

Based on results from the above winner-take-all analysis, the motion that was most preferred was identified at the center of each the three effector-specific (toe movement, hand movement, tongue movement) and inter-effector regions (abdominal movement, eyelid movement, swallowing). The center-most movements were selected to avoid issues with spreading, overlapping activation near the borders of effector-specific and inter-effector regions. For every vertex within the precentral gyrus, the strength of activation was compared between the most preferred of the six motions at that vertex against the activation of the second-most preferred motion. The differences between these activation strengths was taken to be the motion selectivity of that vertex.

### Somatotopic Coactivation

For each region among the six resting state-derived foot, hand, mouth, and inter-effector regions in the precentral gyrus, the average activation was calculated within that region for each motor motion, producing a profile of motor activation strengths for that region. The average activation was also calculated within all CON vertices for each motor motion. To determine the degree to which various motor regions were coactive across motor motions, each foot, hand, mouth, and inter-effector cluster's profile of activation strengths was correlated with that of all other clusters, and with that of the CON. Note: visualization of motor activation maps revealed some striping, suggesting that the Open/Close Mouth and the Bend L Knee conditions were partially contaminated with head motion; as such, these conditions were excluded from analysis, though their inclusion would not change results.

### Action Control and Coordination Task Analyses

Analysis of the motor control task was conducted using within-participant event related designs. For each separate run, a GLM model was constructed in FEAT in which separate regressors described the initiation of 1) planning and 2) execution of each type of movement (4 movements*4 limbs). Each regressor was constructed as a 0-length event convolved with a canonical hemodynamic response, and beta values for each regressor were estimated for every voxel in the brain. These beta value maps for each condition were thus computed for each run and entered into a second-level analysis, in which a t-test across runs contrasted the run-level planning betas against the run-level execution betas. Movement type and dual vs single movement were not considered for the present analyses.

### Human Direct Electrocortical Stimulation Site Mapping

Each stimulation location reported was separately mapped into the MNI-space Conte69 atlas pial cortical surface by identifying the vertex with the minimal Euclidean distance to the stimulation site's MNI coordinates. Movements resulting from each site were classified as “lower extremity”, “upper extremity”, or “face” and colored accordingly (though no lower extremity movements were reported in the displayed left hemisphere).

### Example 2

### Clinical Evidence for Separable Movement Isolation and Action Integration Systems

The Mind-Body Interface includes two important thalamic nuclei that serve as deep brain stimulation (DBS) targets for clinical pathologies: the VIM for tremor (e.g., Essential Tremor, Parkinson's Disease) and the CM for generalized seizures (e.g. Lennox-Gastaut Syndrome). The mechanisms of action underlying these clinical effects are still debated, but it has been proposed that the anti-tremor effects of the VIM may be mediated by its connectivity to the cerebellum, and that the CM's role in arousal may explain its anti-epileptic effects.

Links with the MBI may be critical for the effects of neuromodulation on movement disorders. Tremor and arousal represent important aspects of integrative action control. Physiological tremor (˜10 Hz) is thought to time and coordinate movements, while physiological arousal and CON engagement are observed as goal-directed activity begins.

Many types of tremors are intention- or goal-related. For example, essential tremor is absent or minimal at rest and is brought out by intentional movements. Further, most tremors disappear in sleep. Finally, tremor and generalized seizures are global phenomena, not characterized by somatotopic specificity. Thus, the effects of VIM and CM DBS are consistent with the modulation of different aspects of whole-body action control, movement timing, and arousal.

Parkinson's disease (PD) may be most specifically related to dysfunction of MBI circuitry. PD symptoms cut across motor, physiological and volitional domains (e.g., postural instability, autonomic dysfunction, and reduced self-initiated activity, among many others), mirroring MBI connections to regions relevant for postural control (cerebellar vermis), volition (dACC), and physiological regulation (insula). Work by Clinton Woolsey documented that direct stimulation in M1 of a PD patient temporarily eliminated his whole-body tremor and rigidity. This effect is difficult to explain as a result of stimulation of effector-specific regions, but is fairly straightforward as a consequence of MBI stimulation. Neuronal death in the substantia nigra (SN) is one of the pathophysiological hallmarks of PD. Interestingly, the main target of SN projections is the dorsolateral putamen, which forms part of the Mind-Body Interface. Inter-effector regions are also strongly functionally connected to the cerebellar vermis, important for postural control and known to be structurally connected to M1 in NHP. In addition, cortical projections important for coordinating physiology with action plans (i.e., blood pressure, orthostasis) primarily originate in CON and anterior M1. Thus, if PD is indeed a network disease caused by the retrograde spread of synucleinopathy from the viscera to the brainstem and beyond, a fitting candidate for the network most affected by the resulting degeneration is the Mind-Body Interface.

Brain lesion data further support the existence of dual systems for movement isolation and action integration, with partial redundancy in M1. Motor deficits after middle cerebral artery strokes are unilateral, more severe in most distal effectors, and without significant global organismal control deficits. By contrast, lesions to MBI-linked CON regions (dACC, anterior insula, aPFC) can cause isolated volitional deficits ranging from decreased fluency to abulia to akinetic mutism, with preserved motor abilities but little self-generated movement. Similarly, anterior motor lesions in macaques can spare visually guided movements while selectively disrupting internally generated actions. Interestingly, patients or animals with lesions in effector M1 typically recover gross effector control quickly, with persistent deficits in isolated fine finger movements. More rapid recovery of gross motor abilities may be in part due to proximal functions being taken up by the contralesional MBI circuits, enabled by their bilateral spinal cord connectivity. Persistent deficits may therefore be more likely in functions uniquely supported by the effector-specific circuitry.

In a proof-of-principle patient with extensive bilateral perinatal strokes but typical motor ability, extensive post-stroke reorganization maintained the MBI patterning at the cost of sacrificing part of the already reduced M1 hand area. The top third of M1 was destroyed, and surviving cortex contained an M1 hand area that was ventrally shifted and much smaller than in typical controls. Surprisingly, MBI regions were identified both above and below the surviving effector-specific hand region (FIG. 12F), highlighting the Mind-Body Interface's importance for typical motor ability.

What is claimed is:

1. A system for mapping a network in a brain of a subject, the system comprising at least one processor in communication with at least one memory device, wherein the at least one processor is programmed to:
receive task-based functional magnetic resonance imaging (fMRI) data of a brain of a subject;
receive resting-state fMRI data of the brain;
locate effector-specific regions of the brain and inter-effector regions of the brain based on the task-based fMRI data and the resting-state fMRI data;
identify a network in the brain based on functional connectivity with the inter-effector regions based on the resting-state fMRI data; and
generate one or more maps of the network.

2. The system of claim 1, wherein the at least one processor is further programmed to:
receive the task-based fMRI data acquired during an action planning task with planning phases of movement separate from execution phases of the movement; and
locate the inter-effector regions as regions having higher activation in the planning phases than in the execution phases based on the task-based fMRI data.

3. The system of claim 1, wherein the at least one processor is further programmed to:
locate the inter-effector regions based on functional connectivity with seeds selected from a precentral gyrus of the brain.

4. The system of claim 1, wherein the at least one processor is further programmed to:
receive structural MRI data of the brain; and
verify the inter-effector regions as regions having lower thickness than the effector-specific regions, based on the structural MRI data.

5. The system of claim 1, wherein the at least one processor is further programmed to:
determine functional connectivity of the inter-effector regions with voxels of the brain and functional connectivity of the effector-specific regions with the voxels of the brain based on the resting-state fMRI data; and
identify the network as regions including voxels that have higher functional connectivity with the inter-effector regions than with the effector-specific regions.

6. The system of claim 5, wherein the voxels are selected from cingulo-opercular network.

7. The system of claim 5, wherein the voxels are selected from thalamus.

8. The system of claim 5, wherein the voxels are selected from a postcentral gyrus, insula, putamen, and/or cerebellum.

9. The system of claim 1, wherein the at least one processor is further programmed to:
identify the network based on diffusion-weighted imaging data.

10. A method of identifying a treatment comprising:
receiving task-based functional magnetic resonance imaging (fMRI) data of a brain of a subject;
receiving resting-state fMRI data of a brain of a subject;
locating effector-specific regions of the brain and inter-effector regions of the brain based on the task-based fMRI data and the resting-state fMRI data;
identifying a network based on functional connectivity with the inter-effector regions based on the resting-state fMRI data;
generating one or more maps of the network; and
selecting a treatment based on the one or more maps of the network.

11. The method of claim 10, wherein selecting a treatment further comprises:
selecting a treatment target as a centromedian nucleus of a thalamus based on the one or more maps.

12. The method of claim 10, wherein the method further comprises:
receiving the task-based fMRI data further comprising:
receiving the task-based fMRI data acquired during an action planning task with planning phases of movement separate from execution phases of the movement; and
locating the inter-effector regions as regions having higher activation in the planning phases than in the execution phases based on the task-based fMRI data.

13. The method of claim 10, wherein the method further comprises:
locate the inter-effector regions based on functional connectivity with seeds selected from a precentral gyrus of the brain.

14. The method of claim 10, wherein the method further comprises:
receiving structural MRI data of the brain; and
verifying the inter-effector regions as regions having lower thickness than the effector-specific regions, based on the structural MRI data.

15. The method of claim 10, wherein the method further comprises:
determining functional connectivity of the inter-effector regions with voxels of the brain and functional connectivity of the effector-specific regions with the voxels based on the resting-state fMRI data; and
identifying the network as regions including voxels that have higher functional connectivity with the inter-effector regions than with the effector-specific regions.

16. The method of claim 15, wherein the voxels are selected from cingulo-opercular network.

17. The method of claim 15, wherein the voxels are selected from thalamus, putamen, and/or cerebellum.

18. The method of claim 10, wherein the method further comprises:
mapping a baseline network before the treatment;
mapping a post-treatment network after the treatment; and
estimating an efficacy of the treatment based on changes in one or more maps of the post-treatment network from one or more maps of the baseline network.

19. The method of claim 10, wherein selecting the treatment comprises:
selecting the treatment of a brain disorder that includes neuropsychiatric symptoms, neuropsychiatric disorders, and/or brain injuries.

20. The method of claim 10, wherein the treatment is selected from invasive neuromodulation, non-invasive neuromodulation, and ablative techniques.
